SEQUENCE LISTING (19 sequences)

SEQ ID NO: 1 - protein, 503 aa, Homo Sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 2 - DNA, 1512 bp, Homo Sapiens
atgaccttgggctcccccaggaaaggccttctgatgctgctgatggccttggtgacccagggagaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccagctggccctgatcctgggccccgtgctggccttgctggccctggtggccctgggtgtcctgggcctgtggcatgtccgacggaggcaggagaagcagcgtggcctgcacagcgagctgggagagtccagtctcatcctgaaagcatctgagcagggcgacagcatgttgggggacctcctggacagtgactgcaccacagggagtggctcagggctccccttcctggtgcagaggacagtggcacggcaggttgccttggtggagtgtgtgggaaaaggccgctatggcgaagtgtggcggggcttgtggcacggtgagagtgtggccgtcaagatcttctcctcgagggatgaacagtcctggttccgggagactgagatctataacacagtgttgctcagacacgacaacatcctaggcttcatcgcctcagacatgacctcccgcaactcgagcacgcagctgtggctcatcacgcactaccacgagcacggctccctctacgactttctgcagagacagacgctggagccccatctggctctgaggctagctgtgtccgcggcatgcggcctggcgcacctgcacgtggagatcttcggtacacagggcaaaccagccattgcccaccgcgacttcaagagccgcaatgtgctggtcaagagcaacctgcagtgttgcatcgccgacctgggcctggctgtgatgcactcacagggcagcgattacctggacatcggcaacaacccgagagtgggcaccaagcggtacatggcacccgaggtgctggacgagcagatccgcacggactgctttgagtcctacaagtggactgacatctgggcctttggcctggtgctgtgggagattgcccgccggaccatcgtgaatggcatcgtggaggactatagaccacccttctatgatgtggtgcccaatgaccccagctttgaggacatgaagaaggtggtgtgtgtggatcagcagacccccaccatccctaaccggctggctgcagacccggtcctctcaggcctagctcagatgatgcgggagtgctggtacccaaacccctctgcccgactcaccgcgctgcggatcaagaagacactacaaaaaattagcaacagtccagagaagcctaaagtgattcaatag

SEQ ID NO: 3 - protein, 328 aa, Artificial Sequence
  Recombinant Protein
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1075 bp, Artificial Sequence
  Recombinant DNA
gctagcaccatggatgcaatgaagagagggctctgctgtgtgctgctgctgtgtggagcagtcttcgtttcgcccggcgccgaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcaggggccgccccaccgagttcgtcaaccactactgctgcgacagccacctctgcaaccacaacgtgtccctggtgctggaggccacccaacctccttcggagcagccgggaacagatggccagctggccaccggtggtggaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggccccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaggaattc

SEQ ID NO: 5 - skipped

SEQ ID NO: 6 - protein, 225 aa, Homo Sapiens
  MISC_FEATURE (43)..(43): Xaa can be Asp or Ala
  MISC_FEATURE (100)..(100): Xaa can be Lys or Ala
  MISC_FEATURE (212)..(212): Xaa can be Asn or Ala
THTCPPCPAPELLGGPSVFLFPPKPKDTLMISRTPEVTCVVVXVSHEDPEVKFNWYVDGVEVHNAKTKPREEQYNSTYRVVSVLTVLHQDWLNGKEYKCXVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHXHYTQKSLSLSPGK

SEQ ID NO: 7 - protein, 21 aa, Artificial Sequence
  Recombinant Leader Peptide
MKFLVNVALVFMVVYISYIYA

SEQ ID NO: 8 - protein, 22 aa, Artificial Sequence
  Recombinant Leader Peptide
MDAMKRGLCCVLLLCGAVFVSP

SEQ ID NO: 9 - protein, 21 aa, Artificial Sequence
  Recombinant Leader Peptide
MTLGSPRKGLLMLLMALVTQG

SEQ ID NO: 10 - protein, 429 aa, Homo Sapiens
MCPGALWVALPLLSLLAGSLQGKPLQSWGRGSAGGNAHSPLGVPGGGLPEHTFNLKMFLENVKVDFLRSLNLSGVPSQDKTRVEPPQYMIDLYNRYTSDKSTTPASNIVRSFSMEDAISITATEDFPFQKHILLFNISIPRHEQITRAELRLYVSCQNHVDPSHDLKGSVVIYDVLDGTDAWDSATETKTFLVSQDIQDEGWETLEVSSAVKRWVRSDSTKSKNKLEVTVESHRKGCDTLDISVPPGSRNLPFFVVFSNDHSSGTKETRLELREMISHEQESVLKKLSKDGSTEAGESSHEEDTDGHVAAGSTLARRKRSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 11 - protein, 424 aa, Homo Sapiens
MGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRRNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 12 - protein, 503 aa, Homo Sapiens; identical to SEQ ID NO: 1

SEQ ID NO: 13 - protein, 658 aa, Homo Sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 14 - protein, 625 aa, Homo Sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTREYPRPPQ

SEQ ID NO: 15 - protein, 561 aa, Homo Sapiens
ETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKG

SEQ ID NO: 16 - protein, 582 aa, Homo Sapiens
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 569 aa, Homo Sapiens
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLITGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 563 aa, Homo Sapiens
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLITGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 550 aa, Homo Sapiens
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2014/0193425 A1
(43) Pub. Date: Jul. 10, 2014

(54) TREATMENT OF CANCER WITH ALK1 ANTAGONISTS

(71) Applicant: Acceleron Pharma, Inc., Cambridge, MA (US)

(72) Inventors: John Knopf, Carlisle, MA (US); Ravindra Kumar, Acton, MA (US); Robert S. Pearsall, North Reading, MA (US); Matthew L. Sherman, Newton, MA (US)

(73) Assignee: Acceleron Pharma, Inc., Cambridge, MA (US)

(21) Appl. No.: 14/046,195

(22) Filed: Oct. 4, 2013

Related U.S. Application Data
(60) Provisional application No. 61/710,654, filed on Oct. 5, 2012.

(51) Int. Cl. A61K 39/395; A61K 38/18; C07K 16/40; A61K 38/45; A61K 38/17

(52) U.S. Cl. CPC A61K 39/3955; A61K 38/45; A61K 38/1709; C07K 16/40; A61K 38/1741; A61K 38/1875
USPC 424/158.1; 424/94.3; 424/172.1; 514/13.3; 514/8.8; 435/7.1; 435/7.23

(57) ABSTRACT

Methods for evaluating responsiveness of a subject having cancer to treatment with an activin receptor-like kinase 1 (ALK1) antagonist are provided. Methods for selecting a subject for treatment with an ALK1 antagonist based on the subject being identified as responsive to such treatment are also provided. Some of the diagnostic methods provided herein are based on detecting an ALK1 agonist, e.g., an ALK1 ligand such as BMP9 or BMP10, in a sample obtained from the subject. Diagnostic reagents and kits for determining whether a subject is responsive to treatment with an ALK1 antagonist are also provided.

### CROSS-REFERENCE TO RELATED APPLICATIONS

This application claims benefit under 35 U.S.C. §119(e) of the U.S. Provisional Application No. 61/710,654, filed Oct. 5, 2012, the content of which is incorporated herein by reference in its entirety

### BACKGROUND

Angiogenesis, the process of forming new blood vessels, is critical in many normal and abnormal physiological states. Under normal physiological conditions, humans and animals undergo angiogenesis in specific and restricted situations. For example, angiogenesis is normally observed in wound healing, fetal and embryonic development and formation of the corpus luteum, endometrium and placenta.

Undesirable or inappropriately regulated angiogenesis occurs in many disorders, in which abnormal endothelial growth may cause or participate in the pathological process. For example, angiogenesis participates in the growth of many tumors. Deregulated angiogenesis has been implicated in pathological processes such as rheumatoid arthritis, retinopathies, hemangiomas, and psoriasis. The diverse pathological disease states in which unregulated angiogenesis is present have been categorized as angiogenesis-associated diseases.

Both controlled and uncontrolled angiogenesis are thought to proceed in a similar manner. Capillary blood vessels are composed primarily of endothelial cells surrounded by a basement membrane. Angiogenesis begins with the erosion of the basement membrane by enzymes released by endothelial cells and leukocytes. The endothelial cells, which line the lumen of blood vessels, then protrude through the basement membrane. Angiogenic factors induce the endothelial cells to migrate through the eroded basement membrane. The migrating cells form a “sprout” protruding from the parent blood vessel, where the endothelial cells undergo mitosis and proliferate. Endothelial sprouts merge with each other to form capillary loops, creating the new blood vessel.

Agents that inhibit angiogenesis have proven to be effective in treating a variety of disorders. Avastin™ (bevacizumab), a monoclonal antibody that binds to Vascular Endothelial Growth Factor (VEGF), is used in the treatment of a variety of cancers. Macugen™, an aptamer that binds to VEGF has proven to be effective in the treatment of neovascular (wet) age-related macular degeneration. Antagonists of the SDF/CXCR4 signaling pathway inhibit tumor neovascularization and are effective against cancer in mouse models (Guleng et al. Cancer Res. 2005 Jul. 1; 65(13):5864-71). A variety of so-called multitargeted tyrosine kinase inhibitors, including vandetanib, sunitinib, axitinib, sorafenib, vatalanib, and pazopanib are used as anti-angiogenic agents in the treatment of various tumor types. Thalidomide and related compounds (including pomalidomide and lenalidomide) have shown beneficial effects in the treatment of cancer, and although the molecular mechanism of action is not clear, the inhibition of angiogenesis appears to be an important component of the antitumor effect (see, e.g., Dredge et al. Microvasc Res. 2005 January; 69(1-2):56-63). Although many anti-angiogenic agents have an effect on angiogenesis regardless of the tissue that is affected, other angiogenic agents may tend to have a tissue-selective effect.

It is desirable to have additional compositions and methods for inhibiting angiogenesis associated with disease or disorder, e.g., angiogenesis associated with cancer or a tumor. These include methods and compositions which can inhibit the unwanted growth of blood vessels, either generally or in certain tissues and/or disease states.

### SUMMARY

In some aspects, the disclosure provides activin receptor-like kinase 1 (ALK1) antagonists and the use of such ALK1 antagonists as anti-angiogenic agents in certain subjects. Some aspects of this disclosure also provide antagonists of ALK1 ligands and the use of such ALK1 ligand antagonists as anti-angiogenic agents in certain subjects. methods As described herein, ALK1 is a receptor for bone morphogenetic protein (BMP) ligands, in particular, for BMP9 and BMP10. Signaling mediated by ALK1 and the BMP9/BMP10 ligands is involved in angiogenesis in vivo, and inhibition of this regulatory system has a potent anti-angiogenic effect.

Some aspects of this disclosure provide methods for determining whether a subject having a cancer, e.g., as manifest by a vascularized tumor or a tumor that is associated or dependent on angiogenesis, is responsive to treatment with an ALK1 antagonist, e.g., in that treatment with an ALK1 antagonist will result in a desired clinical effect, such as tumor regression, delay of tumor progression, or inhibition of tumor formation or tumor recurrence.

In some aspects, the disclosure provides methods for evaluating whether a subject is responsive to treatment with an ALK1 antagonist. In some embodiments, the method comprises determining a level of bone morphogenetic protein 9 (BMP9) and/or bone morphogenetic protein 10 (BMP10) in a sample obtained from the subject, and comparing the level of BMP9 and/or BMP10 determined in a sample obtained from the subject to a reference level. In some embodiments, if the level determined in a sample obtained from the subject is higher than the reference level, the subject is identified as responsive to treatment with the ALK1 antagonist. In some embodiments, if the level determined in a sample obtained from the subject is the same as or lower than the reference level, the subject is identified as not responsive to treatment with the ALK1 antagonist. In some embodiments the reference level is a level of BMP9 and/or BMP10 determined in a sample (e.g., tissue or blood) from a healthy subject. In some embodiments the reference level is a level of BMP9 and/or BMP10 determined in sample (e.g., tissue or blood) obtained from the subject at a different time point. In some embodiments the reference level is a level of BMP9 and/or BMP10 expected or observed in a sample obtained from a healthy subject, or an aggregate or average level of BMP9 and/or BMP10 expected or observed in samples from a population of healthy subjects. A healthy subject is a subject who has no signs or symptoms of disease and/or a subject when examined by a medical professional is identified as not having evidence of disease. In some embodiments, the level of BMP9 and/or BMP10 is determined in a sample obtained from the subject comprising or suspected to comprise malignant cells, e.g., tumor cells. In some embodiments, the ALK1 antagonist comprises an ALK1-Fc fusion protein, an ALK1 extracellular domain (ALK-ECD), an antibody or antibody fragment specifically binding ALK1, an antibody or antibody fragment specifically binding an ALK1 ligand, a BMP9 pro-peptide, and/or a BMP10 pro-peptide. In some embodiments, the ALK1 antagonist comprises a polypeptide that is at least 95% identical to the polypeptide provided in SEQ ID NO: 3. In some embodiments, the level of BMP9 and/or BMP10 is determined in a sample obtained from the subject. In some embodiments, the sample is a tissue sample or body fluid sample. In some embodiments, the tissue sample comprises tumor tissue or tumor cells. In some embodiments, the body fluid is blood, plasma, serum, lymph, sputum, cerebrospinal fluid, or urine. In some embodiments, the level of BMP9 and/or BMP10 is determined by measuring the level of a BMP9 and/or BMP10 gene product. In some embodiments, the gene product is a protein or an mRNA. In some embodiments, the treatment with the ALK1 antagonist is a treatment for cancer. In some embodiments, the subject is diagnosed with or is suspected to have a cancer. Cancers are also sometimes referred to as neoplastic disorders. Examples of cancers, or neoplastic disorders, include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, gastric cancer, melanoma, and various types of head and neck cancer, including, for example, cancer of the epiglottis, esophagus, larynx, nasopharynx, soft palate, tongue, and vocal cord, as well as hypopharyngeal cancer and head and neck lymph nodes/lymphadenopathy. In some embodiments, head and neck cancer may affect the squamous epithelium, respiratory epithelium, basal layer, or spinous layer of the epiglottis, esophagus, larynx, nasopharynx, soft palate, tongue, or vocal cord, as well as hypolaryngeal cancer and head and neck lymph nodes/lymphadenopathy. Other examples of neoplastic disorders and related conditions include esophageal carcinomas, thecomas, arrhenoblastomas, endometrial hyperplasia, endometriosis, fibrosarcomas, choriocarcinoma, nasopharyngeal carcinoma, laryngeal carcinomas, hepatoblastoma, Kaposi's sarcoma, skin carcinomas, hemangioma, cavernous hemangioma, hemangioblastoma, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, Wilm's tumor, prostate carcinoma, abnormal vascular proliferation associated with phakomatoses, and Meigs' syndrome. In some embodiments, the cancer is breast cancer, multiple myeloma, cervical cancer, colorectal cancer, endometrial cancer, head and neck cancer (e.g., cancer of the epiglottis, esophagus, larynx, nasopharynx, soft palate, tongue, vocal cord, hypopharynx, and head and neck lymph nodes/lymphadenopathy), liver cancer, lung cancer, malignant carcinoma, malignant glioma, malignant lymphoma, malignant melanoma, ovarian cancer, or pancreatic cancer. In some embodiments, the cancer is resistant to an angiogenesis inhibitor not comprising an ALK1 antagonist. In some embodiments, the method further comprises administering the ALK1 antagonist to the subject.

In some aspects, the disclosure provides methods for selecting a subject having cancer or at risk of developing cancer for treatment with an ALK1 based on the subject having a level of an ALK1 ligand, e.g., of BMP9 and/or BMP10, that is higher than a reference level. In some embodiments, the method comprises selecting the subject for treatment with an ALK1 antagonist on the basis that the subject has a level of an ALK1 antagonist, e.g., of BMP9 and/or BMP10, that is higher than a reference level, and administering the ALK1 antagonist to the subject. In some embodiments, the subject is diagnosed with or is suspected to have a cancer. In some embodiments, the cancer is breast cancer, multiple myeloma, cervical cancer, colorectal cancer, endometrial cancer, head and neck cancer (e.g., cancer of the epiglottis, esophagus, larynx, nasopharynx, soft palate, tongue, vocal cord, hypopharynx, and head and neck lymph nodes/lymphadenopathy), liver cancer, lung cancer, malignant carcinoid, malignant glioma, malignant lymphoma, malignant melanoma, ovarian cancer, or pancreatic cancer. In some embodiments, the cancer is resistant to an angiogenesis inhibitor not comprising an ALK1 antagonist. In some embodiments the reference level is a level of BMP9 and/or BMP10 determined in a sample (e.g., tissue or blood) from a healthy subject. In some embodiments the reference level is a level of BMP9 and/or BMP10 determined in sample (e.g., tissue or blood) obtained from the subject at a different time point. In some embodiments the reference level is a level of BMP9 and/or BMP10 expected or observed in a sample obtained from a healthy subject, or an aggregate or average level of BMP9 and/or BMP10 expected or observed in samples from a population of healthy subjects. A healthy subject is a subject who has no signs or symptoms of disease and/or a subject when examined by a medical professional is identified as not having evidence of disease. In some embodiments, the ALK1 antagonist comprises an agent selected from the group consisting of an ALK1-Fc fusion protein, an ALK1 extracellular domain (ALK-ECD), an antibody or antibody fragment specifically binding ALK1, an antibody or antibody fragment specifically binding an ALK1 ligand, a BMP9 pro-peptide, and a BMP10 pro-peptide. In some embodiments, the ALK1 antagonist comprises a polypeptide that is at least 95% identical to the polypeptide provided in SEQ ID NO: 3. In some embodiments, the method further comprises determining the level of a BMP9 and/or BMP10 gene product in a sample obtained from the subject.

In some aspects, the disclosure provides methods of using ALK1 antagonists for the treatment of certain types of cancers or subjects that have been determined to be responsive to ALK1 antagonist treatment or have been selected for treatment with an ALK1 antagonist based on a diagnostic method provided herein. In some embodiments, the methods provided herein are useful for the treatment of cancers, and in particular of tumors that are vascularized or otherwise require or are associated with angiogenesis, e.g., ALK1-mediated angiogenesis, for example, of breast cancer, multiple myeloma, cervical cancer, colorectal cancer, endometrial cancer, head and neck cancer (e.g., cancer of the epiglottis, esophagus, larynx, nasopharynx, soft palate, tongue, vocal cord, hypopharynx, and head and neck lymph nodes/lymphadenopathy), liver cancer, lung cancer, malignant carcinoma, malignant glioma, malignant lymphoma, malignant melanoma, ovarian cancer, or pancreatic cancer. In some embodiments, the disclosure provided methods of using ALK1 antagonists to inhibit angiogenesis in certain subjects, for example, in subjects having a cancer n elevated ALK1 signaling activity.

Some aspects of this disclosure provide in vitro methods for evaluating responsiveness of a subject to treatment with an activin receptor-like kinase 1 (ALK1) antagonist. In some embodiments, the method comprises determining a level of bone morphogenetic protein 9 (BMP9) and/or bone morphogenetic protein 10 (BMP10) in a sample obtained from the subject; and comparing the level of BMP9 and/or BMP10 determined in the sample to a reference level, wherein if the level determined in the sample is higher than the reference level, the subject is identified as responsive to treatment with the ALK1 antagonist; or if the level determined in the sample is the same or lower than the reference level, the subject is identified as not responsive to treatment with the ALK1 antagonist. In some embodiments, the ALK1 antagonist comprises an agent selected from the group consisting of an ALK1-Fc fusion protein, an ALK1 extracellular domain (ALK-ECD), an antibody or antibody fragment specifically binding ALK1, an antibody or antibody fragment specifically binding an ALK1 ligand, an endoglin ECD antibody, an endoglin ECD, a BMP9 pro-peptide, and a BMP10 pro-peptide. In some embodiments, the ALK1 antagonist comprises a polypeptide that is at least 95% identical to the polypeptide provided in SEQ ID NO: 3. In some embodiments, the level of BMP9 and/or BMP10 is determined in a sample obtained from the subject. In some embodiments, the sample is a tissue sample or body fluid sample. In some embodiments, the tissue sample comprises a tumor tissue or a tumor cell. In some embodiments, the body fluid is blood, plasma, serum, lymph, sputum, cerebrospinal fluid, or urine. In some embodiments, the level of BMP9 and/or BMP10 is determined by measuring the level of a BMP9 and/or BMP10 gene product. In some embodiments, the gene product is a protein or an mRNA. In some embodiments, the treatment with the ALK1 antagonist is a treatment for cancer. In some embodiments, the subject is diagnosed with or is suspected to have a cancer. In some embodiments, the cancer is breast cancer, multiple myeloma, cervical cancer, colorectal cancer, endometrial cancer, head and neck cancer (e.g., cancer of the epiglottis, esophagus, larynx, nasopharynx, soft palate, tongue, vocal cord, hypopharynx, and head and neck lymph nodes/lymphadenopathy), liver cancer, lung cancer, malignant carcinoma, malignant glioma, malignant lymphoma, malignant melanoma, ovarian cancer, or pancreatic cancer. In some embodiments, the cancer is resistant to an angiogenesis inhibitor not comprising an ALK1 antagonist.

Some aspects of this disclosure provide ALK1 antagonists for use in a method for the treatment of a subject having elevated BMP9 and/or BMP10 levels as compared to a reference level. Some aspects of this disclosure provide ALK1 antagonists for use in a method for the treatment of a subject, which subject exhibits a level of BMP9 and/or BMP10 that is higher than a reference level. Some aspects of this disclosure provide ALK1 antagonists for use in the treatment of a subject, wherein the subject is selected for treatment with the ALK1 antagonist on the basis that the subject exhibits a level of BMP9 and/or BMP10 that is higher than a reference level. In some embodiments, the subject has not been diagnosed with a disease or condition that can be treated with the ALK1 antagonist, and wherein the subject is not indicated otherwise for treatment with the ALK1 antagonist. In some embodiments, the subject is diagnosed with or is suspected to have a cancer. In some embodiments, n the cancer is breast cancer, multiple myeloma, cervical cancer, colorectal cancer, endometrial cancer, head and neck cancer (e.g., cancer of the epiglottis, esophagus, larynx, nasopharynx, soft palate, tongue, vocal cord, hypopharynx, and head and neck lymph nodes/lymphadenopathy), liver cancer, lung cancer, malignant carcinoid, malignant glioma, malignant lymphoma, malignant melanoma, ovarian cancer, or pancreatic cancer. In some embodiments, the cancer is resistant to an angiogenesis inhibitor not comprising an ALK1 antagonist. In some embodiments, the ALK1 antagonist comprises an agent selected from the group consisting of an ALK1-Fc fusion protein, an ALK1 extracellular domain (ALK-ECD), an antibody or antibody fragment specifically binding ALK1, an antibody or antibody fragment specifically binding an ALK1 ligand, an endoglin ECD antibody, an endoglin ECD, a BMP9 pro-peptide, and a BMP10 pro-peptide. In some embodiments, the ALK1 antagonist comprises a polypeptide that is at least 95% identical to the polypeptide provided in SEQ ID NO: 3.

Some aspects of this disclosure provide diagnostic kits for evaluating responsiveness of a subject to treatment with an activin receptor-like kinase 1 (ALK1) antagonist. In some embodiments, the kit comprises an agent for detecting a BMP9 and/or BMP10 gene product in a sample; and instructions for detecting and/or quantifying a BMP9 and/or BMP10 gene product. In some embodiments, the gene product is a protein. In some embodiments, the gene product is a transcript. In some embodiments, the agent is a binding agent that specifically binds the BMP9 and/or BMP10 gene product. In some embodiments, the binding agent is an antibody or an antibody fragment that specifically bind the gene product. In some embodiments, the binding agent is a nucleic acid that specifically hybridizes to the gene product. In some embodiments, the kit further comprises a reference sample comprising a known amount of the BMP9 and/or BMP10 gene product. In some embodiments, the sample comprises blood, plasma, serum, urine, cerebrospinal fluid, sputum, lymph, cells, tissue, aspirate, or stool. In some embodiments, the kit further comprises instructions for quantifying the level of BMP9 and/or BMP10.

Other advantages, features, and uses of the inventions disclosed herein will be apparent from the detailed description of certain non-limiting embodiments; the drawings, and the claims.

### BRIEF DESCRIPTION OF THE DRAWINGS

FIG. 1 shows the amino acid sequence for the human Activin Like Kinase 1, ALK1 (SEQ ID NO:1). Single underlining shows the predicted extracellular domain. Double underlining shows the intracellular domain. The signal peptide and the transmembrane domain are not underlined.

FIG. 2 shows the nucleic acid sequence of a human ALK1 cDNA (SEQ ID NO:2). The coding sequence is underlined. The portion encoding the extracellular domain is double underlined.

FIG. 3 shows an example of a fusion of the extracellular domain of human ALK1 to an Fc domain (SEQ ID NO:3). The hALK1-Fc protein includes amino acids 22-120 of the human ALK1 protein, fused at the C-terminus to a linker (underlined) and an IgG1 Fc region. The bottom panel shows a schematic of the dimerized form.

FIG. 4 shows the anti-angiogenic effect of murine ALK1-Fc (“RAP”) and human ALK1-Fc (“ACE”) in an endothelial cell tube forming assay. All concentrations of RAP and ACE reduced the level of tube formation in response to Endothelial Cell Growth Supplement (ECGF) to a greater degree than the positive control, Endostatin.

FIG. 5 shows the anti-angiogenic effect of the human ALK1-Fc fusion in the CAM assay. hALK1-Fc inhibits angiogenesis stimulated by VEGF, FGF and GDF7.

FIG. 6 shows comparative anti-angiogenic effects of murine ALK1-Fc (mALK1-Fc), hALK1-Fc, a commercially available anti-ALK1 monoclonal antibody (Anti-ALK1 mAb) and a commercially available, neutralizing anti-VEGF monoclonal antibody. The anti-angiogenic effect of the ALK1-Fc constructs is comparable to the effects of the anti-VEGF antibody.

FIG. 7 shows the anti-angiogenic effects of hALK1-Fc and the anti-VEGF antibody in vivo. hALK1-Fc and anti-VEGF had comparable effects on angiogenesis in the eye as measured by the mouse corneal micropocket assay.

FIG. 8 shows resolution of hALK1-Fc (SEQ ID NO: 3) and an hALK1-Fc fusion protein from R&D Systems (Minneapolis, Minn.) by Superose 12 10/300 GL Size Exclusion column (Amersham Biosciences, Piscataway, N.J.). The R&D Systems material contains approximately 13% aggregated protein, as shown by the peaks on the left hand side of the graph, as well as some lower molecular weight species. The material of SEQ ID NO:3 is greater than 99% composed of dimers of the appropriate molecular size.

FIG. 9 shows fluorescent signal from luciferase-expressing Lewis lung cancer (LL/2-luc) cells in mice treated with PBS (circles) and mALK1-Fc (triangles). Tumor cells were injected into the tail vein and treatment (PBS or 10 mg/kg mALK1-Fc IP, twice weekly) was initiated on the day of cell administration. PBS-treated mice were sacrificed on day 22 as being moribund. The treatment and control groups each consisted of seven animals (n=7).

FIG. 10 shows the effects of mALK1-Fc on an orthotopic xenograft model using the MDA-MB-231 cell line, a cell line derived from ER− breast cancer cells. At a dose of 30 mg/kg, the mALK1-Fc has a significant growth delaying effect on the xenograft tumor.

FIG. 11 shows the effects of hALK1-Fc on an orthotopic xenograft model using the MCF-7 cell line, a cell line derived from ER+ breast cancer cells. At a dose of 10 or 30 mg/kg, the hALK1-Fc has a significant growth delaying effect on the xenograft tumor.

FIG. 12 shows the efficacy of ALK1-Fc in orthotopic animal models.

FIG. 13 shows representative stainings of BMP9 expression in 29 head and neck tumor samples.

FIG. 14 shows the nucleic acid sequence of SEQ ID NO: 4.

### DETAILED DESCRIPTION

### 1. Overview

ALK1 is a type I cell-surface receptor for the TGF-β superfamily of ligands and is also known as ACVRL1 and ACVRLK1. ALK1 has been implicated as a receptor for TGF-β1, TGF-β3, BMP9, and BMP10 (Marchuk et al., Hum Mol Genet. 2003; Brown et al., J Biol Chem. 2005 Jul. 1; 280(26):25111-8; David et al., Blood. 2007 Mar. 1; 109(5):1953-61.), and Scharpfenecker et al. (J Cell Sci. 2007 Mar. 15; 120(Pt 6):964-72)). ALK1 has been reported to act as an agonist of angiogenesis See, e.g., U.S. Patent Application Publication US2008/0175844 A1 and U.S. Pat. No. 8,158,584, the entire contents of each of which are incorporated herein by reference.

In mice, loss-of-function mutations in ALK1 lead to a variety of abnormalities in the developing vasculature (Oh et al., Proc. Natl. Acad. Sci. USA 2000, 97, 2626-2631; Urness et al., Nat. Genet. 2000, 26, 328-331). In humans, loss-of-function mutations in ALK1 are associated with hereditary hemorrhagic telangiectasia (HHT, or Osler-Rendu-Weber syndrome), in which patients develop arteriovenous malformations that create direct flow (communication) from an artery to a vein (arteriovenous shunt), without an intervening capillary bed. Typical symptoms of patients with HHT include recurrent epistaxis, gastrointestinal hemorrhage, cutaneous and mucocutaneous telangiectases, and arteriovenous malformations (AVM) in the pulmonary, cerebral, or hepatic vasculature.

The present disclosure relates to the discovery that pro-angiogenic ALK1 ligands, e.g., BMP9 and BMP10, are expressed in certain types of cancer that are associated with, or dependent on, angiogenesis, and that angiogenesis in such cancers can be inhibited by blockage or inhibition of pro-angiogenic ALK1 signaling. Some aspects of this disclosure relate to the discovery that inhibition of angiogenesis in such cancers, e.g., by treatment with an anti-angiogenic ALK1 antagonist as provided herein, in turn, results in a clinically beneficial outcome, e.g., a reduction of tumor size, an inhibition or decrease of the rate of tumor growth, a stabilization of the disease state, or an amelioration of a clinical symptom associated with such cancers.

Some aspects of this disclosure provide diagnostic methods for determining whether a subject having a cancer or a tumor is responsive to treatment with an ALK1 antagonist. In some embodiments, the method comprises determining whether the subject, or a tumor, expresses a pro-angiogenic ALK1 ligand, for example, by detecting the presence or a level of the ALK1 ligand, e.g., BMP9 or BMP10, in a sample obtained from the subject. Depending on whether or not the subject or the tumor expresses an ALK1 ligand, or expresses the ligand at or above a certain threshold level, the subject or the tumor is identified as responsive to treatment with an ALK1 antagonist, for example, an ALK1 antagonist provided herein. In some embodiments, the subject is selected for treatment with an ALK1 antagonist based on the outcome of the diagnostic methods provided herein. Diagnostic kits and reagents useful for the detection of ALK1 ligand expression in a subject, e.g., in a sample obtained from a subject, are also provided.

Some aspects of this disclosure provide that angiogenesis can be inhibited in ALK1 antagonist-responsive cancers associated with, or dependent on, angiogenesis by ALK1 antagonists. Some aspects of this disclosure provide methods of treating subjects having a cancer or a type of cancer expressing pro-angiogenic ALK1 ligands, e.g., BMP9 or BMP10, by administering an ALK1 antagonist to the subject. In some embodiments, methods are provided in which an ALK1 antagonist is administered to a subject having cancer based on the subject or the cancer being identified as responsive to ALK1 antagonist treatment.

The terms used in this specification generally have their ordinary meanings in the art, within the context of this disclosure and in the specific context where each term is used. Certain terms are discussed in the specification, to provide additional guidance to the practitioner in describing the compositions and methods disclosed herein and how to make and use them. The scope or meaning of any use of a term will be apparent from the specific context in which the term is used.

### 2. Diagnostic Methods and Compositions

Some aspects of this disclosure provide diagnostic methods for determining whether a subject having a cancer is responsive to treatment with an ALK1 antagonist. In general, a subject responsive to a treatment is a subject in which the treatment will show a desired clinical effect. The term “responsive to treatment with an ALK1 antagonist” as used herein, accordingly, refers to a subject in which administration of the ALK1 antagonist will have a desired effect. For example, a subject having a cancer, e.g., a tumor associated with or dependent on angiogenesis, and identified to be responsive to treatment with an ALK1 antagonist, e.g., by a diagnostic method provided herein, is a subject that benefits clinically from administration of the ALK1 antagonist. For example, the subject may benefit from administration of an ALK1 antagonist in that administration of an effective amount of the ALK1 antagonist may result in one or more of a reversal, an inhibition, or a delay in tumor development, tumor formation, tumor growth, tumor vascularization, tumor angiogenesis tumor survival, tumor progression, tumor recurrence, or metastasis.

Some of the diagnostic methods provided herein comprise determining whether the subject, or a tumor in a subject, expresses a pro-angiogenic ALK1 ligand. In some embodiments, determining expression of an ALK1 ligand includes detecting the presence or a level of the ALK1 ligand, e.g., BMP9 or BMP10, in the subject or in a sample obtained from the subject. Any assay suitable for detecting an ALK1 ligand may be employed in the diagnostic methods provided herein. If the ALK1 ligand to be detected is a protein or peptide ligand, e.g., BMP9 or BMP10, then any protein or peptide detection assay may be employed. Suitable protein detection assays include, but are not limited to, immunohistochemistry (IHC) assays, antibody staining assays, assays that include staining of the ALK1 ligand with a specific binding agent, Western Blot, protein arrays, mass spectrometry, ELISA assays, and cell based assays. Such methods are well known to those in the art, and so are reagents useful for detection of ALK1 ligands, e.g., of BMP9 and BMP10. See, e.g., R&D Systems catalog #MAB3209 (human/mouse BMP9 antibody), R&D Systems catalog #MAB2926 (human/mouse BMP10 antibody).

In addition to analytical assays aimed at directly detecting a molecule, e.g., an ALK1 ligand, for example, by staining, detecting, and quantifying the molecule, cell based assays may also be used to quantify the levels of multiple BMPs present in a sample as described by Herrera and Inman, A rapid and sensitive bioassay for the simultaneous measurement of multiple bone morphogenetic proteins, BMC Cell Biology 2009, 10:20, the entire contents are incorporated herein by reference. Additionally, multiple analytical assays incorporating automated IHC methods with computer-based programs designed specifically for quantitative IHC analysis have been developed, as reviewed by Cregger et al., Immunohistochemistry and Quantitative Analysis of Protein Expression, Arch Pathol Lab Med 2006 July; 130(7):1026-30, and as exemplified in U.S. Pat. Nos. 8,068,988 and 8,114,615, the entire contents of each of which are incorporated herein by reference. Such assays and methods are suitable for the detection of ALK1 ligands, and particularly suitable assays and methods include, for example, BLISS and IHCscore of Bacus Laboratories, Inc (Lombard, Ill.); ACIS of Clarient, Inc (San Juan Capistrano, Calif.); iVision and GenoMx of BioGenex (San Ramon, Calif.); ScanScope of Aperio Technologies (Vista, Calif.); Ariol SL-50 of Applied Imaging Corporation (San Jose, Calif.); LSC Laser Scanning Cytometer of CompuCyte Corporation (Cambridge, Mass.); and AQUA of HistoRx Inc (New Haven, Conn.). Additional suitable methods for the detection of ALK1 ligands are well known to those of skill in the art, and include, but are not limited to, the detection methods for proteins and nucleic acids described in Sambrook, Joseph. & Russell, David W. & Cold Spring Harbor Laboratory. (2001). Molecular cloning: a laboratory manual. Cold Spring Harbor, N.Y.: Cold Spring Harbor Laboratory, ISBN 0879695773, the entire contents of which are incorporated herein by reference. Additional reagents useful for the detection of ALK1 ligands will be apparent to those of skill in the art and the disclosure is not limited in this respect.

In some embodiments, a subject or a tumor is identified as responsive to treatment with an ALK1 antagonist, for example, an ALK1 antagonist provided herein, depending on whether or not the subject or the tumor expresses an ALK1 ligand, or expresses the ligand at or above a certain threshold level. In some embodiments, the level of expression of the ALK1 ligand, e.g., of BMP9 or BMP10, is quantified and compared to a reference level. In some embodiments, the subject is identified as responsive to treatment with the ALK1 antagonist if the level determined in a sample obtained from the subject or the tumor is higher than the reference level. In some embodiments, the subject is identified as not responsive to treatment with the ALK1 antagonist if the level determined in a sample obtained from the subject or the tumor is equal to or lower than the reference level.

In some embodiments, the reference level is an expression level of the respective ALK1 ligand in healthy tissue. For example, in some embodiments, the reference level is a level of the respective ALK1 ligand, e.g., BMP9 or BMP10, in healthy tissue obtained from the subject, e.g., of healthy tissue of the same type as the tissue the tumor is found in or originates from. For example, if a subject presents with lung cancer, the method for evaluating the responsiveness of the subject to treatment with an ALK1 antagonist may include obtaining a biopsy of the cancerous lung tissue and of healthy lung tissue from the subject, determining the level of a pro-angiogenic ALK1 ligand (e.g., BMP9, or BMP10) in the cancerous tissue and in the healthy tissue, and comparing the level determined in the cancerous tissue to the level determined in the healthy tissue (the reference level in this case). If the level of the ALK1 ligand in the cancerous tissue is found to be higher than the reference level, then the tumor or the subject are determined to be responsive to treatment with an ALK1 antagonist. In some embodiments, the ALK1 antagonist is then administered to the subject in an effective amount to treat the lung cancer.

In some embodiments, the reference level is a level of the respective ALK1 ligand determined in tissue obtained from the subject at a different time point. For example, in some embodiments, a subject diagnosed with or suspected to have a tumor may be monitored over time for signs of aberrant angiogenesis, e.g., aberrant ALK1-mediated angiogenesis, as a proxy for onset of tumorigenesis or tumor growth or for tumor recurrence after a clinical intervention targeted to eliminate the tumor or decrease tumor burden in the subject. In some embodiments, an increase of the level of an ALK1 ligand (e.g., ALK1, BMP9, or BMP10) over time in the subject or the tissue being monitored is indicative of tumor onset, growth, or recurrence, and is also indicative of the tumor or the subject being responsive to ALK1 antagonist treatment.

In other embodiments, the reference level is a level of the respective ALK1 ligand (e.g., ALK1, BMP9, or BMP10) expected or observed in healthy tissue or in tissue obtained from a healthy subject. This type of reference level may be determined by obtaining healthy tissue or tissue from a healthy subject and assaying the level of the respective ALK1 ligand in parallel to the tissue from the subject in question. Alternatively, the level may be determined by analyzing the levels found in healthy tissues or in healthy subjects in the past, and calculating an aggregate level from those levels. Aggregate levels may be average or median levels, or levels based on a plurality of measured or observed levels. Additional appropriate reference level will be apparent to those of skill in the art, and the disclosure is not limited in this respect.

In some embodiments, the terms “higher” and “lower” as well as the terms “increase” and “decrease” and the term “elevated” in the context of levels of ALK1 ligands detected or observed in a tissue or a subject as compared to reference levels, refer to a difference in the measured or observed levels as compared to the reference levels. In preferred embodiments, the difference referred to is a statistically significant reference. Appropriate statistical tests for determining whether a difference is significant will be apparent to those of skill in the art and include, without limitation, T-tests and ANOVA tests. Additional appropriate statistical tests for significance will be apparent to those of skill in the art. In some embodiments, an increased or decreased level of an ALK1 ligand observed in a sample as compared to a reference level is present if the level in the sample is at least 1.5-fold, at least 2-fold, at least 3-fold, at least 4-fold, at least 5-fold, at least 10-fold, at least 20-fold, at least 30-fold, at least 40-fold, at least 50-fold, at least 100-fold, at least 200-fold, at least 300-fold, at least 400-fold, at least 500-fold, or at least 1000-fold increased or decreased, respectively, as compared to the reference level, optionally with a significance of p<0.05, p<0.01, p<0.005, p<0.001, p<0.005, or p<0.001. In some embodiments, an increased level of an ALK1 ligand observed in a sample as compared to a reference level is present if the level in the sample is at least 20%, at least 25%, at least 30%, at least 40%, at least 50%, at least 60%, at least 70%, at least 75%, at least 80%, at least 90%, at least 100%, at least 150%, at least 200%, at least 250%, at least 300%, at least 400%, at least 500%, at least 1000%, at least 2000%, at least 2500%, at least 10000%, or at least 100000% increased as compared to the reference level, optionally with a significance of p<0.05, p<0.01, p<0.005, p<0.001, p<0.005, or p<0.001. In some embodiments, a decreased level of an ALK1 ligand observed in a sample as compared to a reference level is present if the level in the sample is less than 80%, less than 75%, less than 70%, less than 60%, less than 50%, less than 40%, less than 30%, less than 25%, less than 20%, less than 10%, less than 5%, less than 2.5%, less than 2%, less than 1%, less than 0.1%, or less than 0.01%, of the reference level, optionally with a significance of p<0.05, p<0.01, p<0.005, p<0.001, p<0.005, or p<0.001.

In some embodiments, the ALK1 ligand is detected in a sample obtained from a subject. In some embodiments, the sample is a tissue sample or body fluid sample. In some embodiments, the sample is a tissue sample, for example, a sample of healthy or diseased tissue. In some embodiments, the sample is a body fluid sample, for example, a blood, plasma, serum, lymph, sputum, cerebrospinal fluid, or urine sample. In some embodiments, the sample comprises or is suspected to comprise tumor tissue or tumor cells. In some embodiments, the sample comprises breast tissue, bone marrow, cervical tissue, colorectal tissue, endometrial tissue, tissue typically affected by head and neck cancer (for example, epiglottis, esophagus, larynx, nasopharynx, soft palate, tongue, vocal cord, hypopharynx, and head and neck lymph nodes), liver tissue, lung tissue, brain tissue, lymph node tissue, skin tissue, connective tissue, ovarian tissue, or pancreatic tissue. In some embodiments, the tissue sample comprises malignant cells or tumor tissue. In some embodiments, the tissue sample is a tumor biopsy sample. In some embodiments, methods that involve determining a level of an ALK1 agonist, e.g., of an ALK1 ligand such as BMP9 or BMP10, in a sample from a subject include obtaining the sample from the subject. Without wishing to be bound by any particular theory, it is believed that some tumors produce soluble ALK1 ligands that act on the hosts vasculature to induce angiogenesis resulting in tumor vascularization. Some aspects of this disclosure relate to the surprising recognition that that some soluble ALK1 ligands produced by tumors, e.g., BMP9 and BMP10, can be detected at the site of origin, e.g., in tumor tissue, before they enter systemic circulation. Some aspects of this disclosure further relate to the recognition that, while the amount of some growth factors shed from tumors is too small to be detected systemically on the background of normal endogenous production of the growth factor, some ALK1 ligands can be detected in samples taken from a location remote from the tumor site. Accordingly, in some embodiments, an ALK1 ligand is detected in a tumor sample or a sample derived from a tumor. In other embodiments, an ALK1 ligand is detected in a body fluid sample, e.g., a blood, plasma, serum, lymph, sputum, cerebrospinal fluid, or urine sample.

In some embodiments, the subject is selected for treatment with an ALK1 antagonist based on the outcome of the diagnostic methods provided herein. For example, if the subject or a tumor in the subject is identified as responsive to treatment with an ALK1 antagonist, then the subject is selected to receive ALK1 antagonist treatment, e.g., in the form of administering an effective amount of an ALK1 antagonist described herein. In some embodiments, if the subject or a tumor in the subject is identified as not responsive to treatment with an ALK1 antagonist, then the subject is selected to not receive ALK1 antagonist treatment.

### 3. Therapeutic Methods and Compositions

Some aspects of this disclosure provide methods of treating cancer in a subject by administering to the subject an effective amount of an ALK1 antagonist. In some embodiments, the disclosure provides methods that include evaluating responsiveness of a subject to treatment with an ALK1 antagonist and subsequently administering an ALK1 antagonist to a subject if the subject has been identified to be responsive to ALK1 antagonist treatment. In some embodiments, the methods include determining whether the subject exhibits aberrant angiogenesis, e.g., aberrant ALK1-mediated angiogenesis, or, in some embodiments, an increased level of angiogenesis, or an aberrant level of a signaling molecule that is part of the ALK1 regulatory system and that is associated with an aberrant pro-angiogenic state (e.g., an overabundance of a pro-angiogenic ALK1 ligand or of ALK1. In some embodiments, such methods include determining a level of a pro-angiogenic ALK1 agonist in a sample obtained from the subject. The ALK1 agonist, in some embodiments, is ALK1, BMP9, or BMP10.

As used herein, the terms “treatment”, “treating”, and “therapy” refer to therapeutic treatment and prophylactic, or preventative manipulations, or manipulations which reverse, inhibit, or delay tumor development, tumor formation, tumor growth, tumor vascularization, tumor survival, tumor progression, tumor recurrence, or metastasis; reduce the severity of a neoplastic or malignant proliferative disease, e.g., of cancer; and/or ameliorate a symptom associated with a neoplastic or malignant proliferative disease. Tumor development, tumor formation, tumor growth, tumor vascularization, tumor survival, tumor progression, tumor recurrence, and metastasis can be measured by methods known to the skilled artisan, for example, methods described in the Response Evaluation Criteria in Solid Tumors (RECIST) Guidelines (see Therasse et al., Journal of the National Cancer Institute 2000, 92(3):205-213; Eisenhauer et al., European Journal of Cancer 2009, 45:228-247; the entire contents of each of which are incorporated herein by reference). Thus, the terms denote that a beneficial clinical result has been conferred on a subject having cancer, or carrying a tumor or with the potential to develop such disorder. In some embodiments, for example, treatment of a subject having a cancer with an ALK1 antagonist as described herein results in stable disease, lack of disease progression, or regression of disease (e.g., shrinkage of a tumor in the subject by at least 10%, at least 20%, at least 25%, at least 30%, at least 40%, at least 50%, at least 60%, at least 70%, at least 75%, at least 80%, at least 90%, or at least 98%, by mass or volume), according to RECIST guidelines. Furthermore, the term “treatment” is defined as a clinical intervention, e.g., the administration of an agent (e.g., a therapeutic agent or a therapeutic composition comprising an ALK1 antagonist) to a subject, or an isolated tissue or cell obtained from a subject, who has a disease, a symptom of disease, or a predisposition toward a disease, with the purpose to improve the clinical condition of the subject, e.g., with the purpose to cure, heal, alleviate, relieve, remedy, ameliorate, or otherwise positively affect the disease, the symptoms of disease or the predisposition toward disease.

As used herein, a “therapeutic agent” refers to any substance or combination of substances that can be used in the treatment of a disease, e.g., An agent that inhibits tumor vascularization or tumor angiogenesis. Accordingly, a therapeutic agent includes, but is not limited to, the ALK1 antagonists provided herein.

Some aspects of this disclosure describe types of cancers that are particularly suitable for treatment with an ALK1 antagonist. These are typically cancers and tumors that are vascularized, and rely on or require angiogenesis for proliferation, survival, and growth. The terms “cancer” and “cancerous” refer to, or describe a physiological condition that is typically characterized by unregulated cell growth/proliferation. Cancers are also sometimes referred to as neoplastic disorders. Examples of cancers, or neoplastic disorders, include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, gastric cancer, melanoma, and various types of head and neck cancer, including, for example, cancer of the epiglottis, esophagus, larynx, nasopharynx, soft palate, tongue, and vocal cord, as well as hypopharyngeal cancer and head and neck lymph nodes/lymphadenopathy. In some embodiments, head and neck cancer may affect the squamous epithelium, respiratory epithelium, basal layer, or spinous layer of the epiglottis, esophagus, larynx, nasopharynx, soft palate, tongue, or vocal cord, as well as hypopharyngeal cancer and head and neck lymph nodes/lymphadenopathy. Other examples of neoplastic disorders and related conditions include esophageal carcinomas, thecomas, arrhenoblastomas, endometrial hyperplasia, endometriosis, fibrosarcomas, choriocarcinoma, nasopharyngeal carcinoma, laryngeal carcinomas, hepatoblastoma, Kaposi's sarcoma, skin carcinomas, hemangioma, cavernous hemangioma, hemangioblastoma, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, Wilm's tumor, prostate carcinoma, abnormal vascular proliferation associated with phakomatoses, and Meigs' syndrome. In some embodiments, the cancer is breast cancer, multiple myeloma, cervical cancer, colorectal cancer, endometrial cancer, head and neck cancer, liver cancer, lung cancer, malignant carcinoma, malignant glioma, malignant lymphoma, malignant melanoma, ovarian cancer, or pancreatic cancer. In some embodiments, the cancer is resistant to an angiogenesis inhibitor not comprising an ALK1 antagonist. In some embodiments, the method further comprises administering the ALK1 antagonist to the subject. A cancer that is particularly amenable to treatment with the ALK1 antagonists described herein may be characterized by one or more of the following: the cancer has angiogenic activity, an elevated level of at least one ALK1 agonist detectable in the tumor or the serum, e.g., by detecting increased BMP9 or BMP10 expression levels or biological activity, is metastatic or at risk of becoming metastatic, or any combination thereof.

In some embodiments, the methods of treating cancer provided herein include administering to the subject an effective amount of an ALK1 antagonist if the subject is found to exhibit angiogenesis associated with the cancer, including, but not limited to ALK1-mediated angiogenesis. In some embodiments, the method comprises comparing the level of the pro-angiogenic ALK1 agonist (e.g., of BMP9, BMP10, or ALK1) determined in the subject, or in a sample obtained from the subject, to a reference level. In some embodiments, if the level of the pro-angiogenic ALK1 agonist (e.g., of BMP9, BMP10, or ALK1) determined in a sample obtained from the subject is higher than the reference level, the subject is identified as responsive to treatment with the ALK1 antagonist. In some embodiments, if the level pro-angiogenic ALK1 agonist (e.g., of BMP9, BMP10, or ALK1) determined in a sample obtained from the subject is the same or lower than the reference level, the subject is identified as not responsive to treatment with the ALK1 antagonist. In some embodiments, the method includes administering an effective amount of an ALK1 antagonist to the subject, if the subject is found to be responsive to treatment with the ALK1 antagonist.

The disclosure also provides methods of inhibiting or preventing growth of a vascularized tumor, or of a tumor that requires angiogenesis to proliferate, by contacting the tumor with an effective amount of an ALK1 antagonist. Some of the methods provided herein include evaluating responsiveness of the tumor to treatment with an ALK1 antagonist. Accordingly, methods are provided herein that include evaluating responsiveness of a vascularized tumor or a tumor requiring angiogenesis to proliferate to treatment with an ALK1 antagonist and, if the tumor is found to be responsive, contacting the tumor with an effective amount of an ALK1 antagonist. In some embodiments, the method includes determining whether the tumor exhibits angiogenesis, for example, but not limited to, ALK1-mediated angiogenesis, or, in some embodiments, an increased level of angiogenesis, or expresses an aberrant level of a signaling molecule that is part of the ALK1 regulatory system and that is associated with a pro-angiogenic state (e.g., an overabundance of a pro-angiogenic ALK1 ligand or of ALK1. Typically, such methods include obtaining a sample comprising a tumor, or tumor cells, e.g., a biopsy sample from a subject or a cell culture derived from a tumor. In some embodiments, such methods include determining a level of a pro-angiogenic ALK1 agonist in the tumor. The ALK1 agonist, in some embodiments, is ALK1, BMP9, or BMP10.

In some embodiments, the method includes contacting the tumor with an effective amount of an ALK1 antagonist if the tumor is found to exhibit angiogenesis, for example, but not limited to, ALK1-mediated angiogenesis. In some embodiments, the method comprises comparing the level of the pro-angiogenic ALK1 agonist (e.g., of BMP9, BMP10, or ALK1) determined in the tumor to a reference level. In some embodiments, if the level of the pro-angiogenic ALK1 agonist (e.g., of BMP9, BMP10, or ALK1) determined in the tumor is higher than the reference level, the tumor is identified as responsive to treatment with the ALK1 antagonist. In some embodiments, if the level pro-angiogenic ALK1 agonist (e.g., of BMP9, BMP10, or ALK1) determined in the tumor is the same or lower than the reference level, the tumor is identified as not responsive to treatment with the ALK1 antagonist. In some embodiments, the method includes contacting the tumor with an effective amount of an ALK1 antagonist, if the tumor is found to be responsive to treatment with the ALK1 antagonist. In some embodiments, the contacting is in vivo, for example, by administering the ALK1 antagonist to a subject carrying a tumor that is responsive to treatment with an ALK1 antagonist. In some embodiments, the contacting is in vitro, for example, by contacting a tumor biopsy or a cell culture derived therefrom with an ALK1 antagonist in vitro.

In some embodiments, the reference level used for determining whether or not a level of a pro-angiogenic ALK1 signaling molecule (e.g., ALK1, BMP9, or BMP10) is elevated in a subject or a tumor, is a level of the respective pro-angiogenic ALK1 signaling molecule determined in healthy tissue. For example, in some embodiments that involve evaluating the responsiveness of a subject or a tumor in a subject to treatment with an ALK1 antagonist, the reference level is a level of the respective ALK1 signaling molecule determined in healthy tissue obtained from the subject, e.g., of healthy tissue of the same type as the tissue the tumor is found in or originates from. For example, if a subject presents with lung cancer, the method may include taking a biopsy of the cancerous lung tissue and of healthy lung tissue, determining the level of a pro-angiogenic ALK1 signaling molecule (e.g., ALK1, BMP9, or BMP10) in the cancerous tissue and in the healthy tissue, and comparing the level determined in the cancerous tissue to the level determined in the healthy tissue (the reference level in this case). If the level of a pro-angiogenic signaling molecule in the cancerous tissue is found to be higher than the reference level, then the tumor or the subject are determined to be responsive to treatment with an ALK1 antagonist. In some embodiments, the ALK1 antagonist is then administered to the subject in an effective amount to treat the lung cancer.

In some embodiments, the reference level is a level of the respective ALK1 signaling molecule determined in tissue obtained from the subject at a different time point. For example, in some embodiments, a subject diagnosed with or suspected to have a tumor may be monitored over time for signs of aberrant ALK1-mediated angiogenesis, e.g., as a proxy for onset of tumorigenesis or tumor growth or for tumor recurrence after a clinical intervention targeted to eliminate the tumor or decrease tumor burden in the subject. In some embodiments, an increase of the level of a pro-angiogenic ALK1 agonist (e.g., ALK1, BMP9, or BMP10) over time in the subject or the tissue being monitored is indicative of tumor onset, growth, or recurrence, and is also indicative of the tumor or the subject being responsive to ALK1 antagonist treatment. In other embodiments, the reference level is a level of the respective pro-angiogenic ALK1 agonist (e.g., ALK1, BMP9, or BMP10) expected or observed in healthy tissue or in tissue obtained from a healthy subject. This type of reference level may be determined by obtaining healthy tissue or tissue from a healthy subject and assaying the level of the respective ALK1 agonist in parallel to the tissue from the subject in question. Alternatively, the level may be determined by analyzing the levels found in healthy tissues or in healthy subjects in the past, and calculating an aggregate level from those levels. Aggregate levels may be average or median levels, or levels based on a plurality of measured or observed levels. Additional appropriate reference level will be apparent to those of skill in the art, and the disclosure is not limited in this respect.

In some embodiments, the terms “higher” and “lower” as well as the terms “increase” and “decrease” in the context of levels of ALK1 agonists measured or observed in tissues or subject as compared to reference levels refer to a difference in the measured or observed levels as compared to the reference levels. In preferred embodiments, the difference referred to is a statistically significant reference. Appropriate statistical tests for determining whether a difference is significant will be apparent to those of skill in the art and include, without limitation, T-tests and ANOVA tests. Additional appropriate statistical tests for significance will be apparent to those of skill in the art. In some embodiments, a statistically significant difference in an observed level and a reference level is a level that is at least 1.5-fold, at least 2-fold, at least 3-fold, at least 4-fold, at least 5-fold, at least 10-fold, at least 20-fold, at least 30-fold, at least 40-fold, at least 50-fold, at least 100-fold, at least 200-fold, at least 300-fold, at least 400-fold, at least 500-fold, or at least 1000-fold increased, optionally with a significance of p<0.05, p<0.01, p<0.005, p<0.001, p<0.005, or p<0.001.

In the context of determining whether or not a subject or a tumor is responsive to ALK1-antagonist treatment, levels of ALK1 agonists (e.g., of ALK1, BMP9, or BMP10) are typically determined in a sample obtained from a subject or from the tumor. In some embodiments, methods that involve determining a level of an ALK1 agonist in a sample from a subject include obtaining the sample from the subject. In some embodiments, the sample is a tissue sample or body fluid sample. In some embodiments, the sample is a tissue sample, for example, a sample of healthy or diseased tissue. In some embodiments, the sample is a body fluid sample, for example, a blood, plasma, serum, lymph, sputum, cerebrospinal fluid, or urine sample. In some embodiments, the sample comprises or is suspected to comprise tumor tissue or tumor cells. In some embodiments, the sample comprises breast tissue, bone marrow, cervical tissue, colorectal tissue, endometrial tissue, tissue typically affected by head and neck cancer, liver tissue, lung tissue, brain tissue, lymph node tissue, skin tissue, connective tissue, ovarian tissue, or pancreatic tissue.

In some embodiments, the term “determining” in the context of the presence or a level of a molecule, e.g., an ALK1 ligand or an ALK1 agonist, refers to performing an analytical assay to detect the presence and/or a level of the molecule. For example, a level of an ALK1 agonist (e.g., ALK1, BMP9, or BMP10) can be determined in a sample obtained from a subject by subjecting the sample to an analytical assay suitable to measure or detect the presence and/or a level of a product of a gene encoding the respective ALK1 agonist, e.g., a level of an ALK1, BMP9, or BMP10 gene product in the sample. A gene product may be a nucleic acid, e.g. a transcript, or a protein or polypeptide. Analytical assays and methods for measuring and quantifying the level of a gene product are well known to those of skill in the art and include, without limitation, western blot, RT-PCR, northern blot, quantitative and qualitative sequencing methods, mass spectrometry, FACS assays, immunohistochemistry (IHC) assays, antibody staining assays, protein arrays, ELISA assays, and cell based assays. Such methods are well known to those in the art, and so are reagents useful for detection of ALK1 agonists, e.g., of BMP9 and BMP10. See, e.g., U.S. Pat. No. 5,932,216, U.S. patent application Ser. No. 10/366,345, and R&D Systems catalog #MAB3209 (human/mouse BMP9 antibody), R&D Systems catalog #MAB2926 (human/mouse BMP10 antibody), the entire contents of each of which are incorporated herein by reference. In addition to analytical assays aimed at directly detecting a molecule, e.g., an ALK1 agonist, for example, by staining, detecting, and quantifying the respective molecule, e.g., by western blot, northern blot, or mass spec, cell based assays may also be used to quantify the levels of multiple BMPs present in a sample, as described, e.g., by Herrera and Inman, A rapid and sensitive bioassay for the simultaneous measurement of multiple bone morphogenetic proteins, BMC Cell Biology 2009, 10:20, the entire contents are incorporated herein by reference. Additionally, multiple analytical assays incorporating automated IHC methods with computer-based programs designed specifically for quantitative IHC analysis have been developed, as reviewed by Cregger et al., Immunohistochemistry and Quantitative Analysis of Protein Expression, Arch Pathol Lab Med 2006 July; 130(7):1026-30, and as exemplified in U.S. Pat. Nos. 8,068,988 and 8,114,615, the entire contents of each of which are incorporated herein by reference. Such assays and methods are suitable for the detection of ALK1 ligands, and particularly suitable assays and methods include, for example, BLISS and IHCscore of Bacus Laboratories, Inc (Lombard, Ill.); ACIS of Clarient, Inc (San Juan Capistrano, Calif.); iVision and GenoMx of BioGenex (San Ramon, Calif.); ScanScope of Aperio Technologies (Vista, Calif.); Ariol SL-50 of Applied Imaging Corporation (San Jose, Calif.); LSC Laser Scanning Cytometer of CompuCyte Corporation (Cambridge, Mass.); and AQUA of HistoRx Inc (New Haven, Conn.). Additional suitable methods for the detection of ALK1 ligands are well known to those of skill in the art, and include, but are not limited to, the detection methods for proteins and nucleic acids described in Sambrook, Joseph. & Russell, David W. & Cold Spring Harbor Laboratory. (2001). Molecular cloning: a laboratory manual. Cold Spring Harbor, N.Y.: Cold Spring Harbor Laboratory, ISBN 0879695773, the entire contents of which are incorporated herein by reference. Some examples of suitable detection reagents and of analytical assays for the detection and/or quantification of ALK1 agonists, including, but not limited to, BMP9 and BMP10, are described herein, e.g., in the Examples section.

In some embodiments, the level of an ALK1 agonist in a sample is determined by assaying a biological activity of the respective ALK1 agonist. Some suitable assays, including, but not limited to, CAM assays, are described herein, and additional suitable methods and analytical assays for assessing and/or quantifying the biological activity of ALK1 agonists, e.g., of BMP9 or BMP10, are known to those of skill in the art.

Some aspects of this disclosure provide methods of treating a cancer in a subject determined to be responsive to an ALK1 antagonist. Some aspects of this disclosure provide methods of treating a tumor determined to be responsive to an ALK1 antagonist. In some embodiments, the cancer or the tumor is resistant to an angiogenesis inhibitor not comprising an ALK1 antagonist, such as a VEGF inhibitor or a PDGF inhibitor. In some embodiments, methods of treating cancer are provided that include selecting a subject for treatment with an ALK1 antagonist based on a determination that the subject has an elevated level of an ALK1 antagonist as compared to a reference level. In some embodiments, the methods of treating a cancer or a tumor include administering an ALK1 antagonist to the subject or contacting the tumor with an ALK1 antagonist. In some embodiments, the subject is diagnosed with or is suspected to have a cancer. In some embodiments, the cancer is breast cancer, multiple myeloma, cervical cancer, colorectal cancer, endometrial cancer, head and neck cancer, liver cancer, lung cancer, malignant carcinoma, malignant glioma, malignant lymphoma, malignant melanoma, ovarian cancer, or pancreatic cancer. In some embodiments, the cancer or tumor is resistant to an angiogenesis inhibitor not comprising an ALK1 antagonist, such as a VEGF inhibitor or a PDGF inhibitor. In some embodiments, the subject has been treated with an angiogenesis inhibitor not comprising an ALK1 antagonist, but has retained a tumor mass or tumor burden requiring further treatment.

In some embodiments, the ALK1 antagonist administered to the subject or contacted to the tumor comprises an ALK1 antagonist described herein, for example, an ALK1-ECD protein, an ALK1-Fc fusion protein, an antibody or antibody fragment specifically binding ALK1, an antibody or antibody fragment specifically binding an ALK1 ligand, a BMP9 pro-peptide, or a BMP10 pro-peptide. In certain embodiments of methods of treating a cancer or a tumor, two or more ALK1 antagonists are administered, either together (simultaneously) or at different times (sequentially). For example, in some embodiments, an ALK1-Fc fusion protein may be administered together with an antibody specifically binding ALK1. In addition, ALK1 antagonists are administered, in some embodiments, in combination with an additional compound for treating cancer or for inhibiting angiogenesis, e.g., with a chemotherapeutic agent, a cytotoxic agent, a cytostatic agent, or an angiogenesis inhibitor.

Suitable compounds for treating cancer and for inhibiting angiogenesis that can be administered in combination with ALK1 antagonists as described herein are known in the arts, including some of those listed herein and, e.g., listed by Carmeliet and Jain, Nature 407:249-257 (2000); Ferrara et al., Nature Reviews: Drug Discovery, 3:391-400 (2004); and Sato Int. J. Clin. Oncol, 8:200-206 (2003). See also, U.S. Patent Application US20030055006. In one embodiment, an ALK1 antagonist as provided herein is used in combination with an anti-VEGF neutralizing antibody (or fragment) and/or another VEGF antagonist or a VEGF receptor antagonist including, but not limited to, for example, soluble VEGF receptor (e.g., VEGFR-I, VEGFR-2, VEGFR-3, neuropillins (e.g., NRP1, NRP2)) fragments, aptamers capable of blocking VEGF or VEGFR, neutralizing anti-VEGFR antibodies, low molecule weight inhibitors of VEGFR tyrosine kinases (RTK), antisense strategies for VEGF, ribozymes against VEGF or VEGF receptors, antagonist variants of VEGF; and any combinations thereof. Alternatively, or additionally, two or more angiogenesis inhibitors may optionally be co-administered to the patient in addition to VEGF antagonist and other agent. In certain embodiment, one or more additional therapeutic agents, e.g., anti-cancer agents such as chemotherapeutic agents, cytostatic and cytotoxic agents, can be administered in combination with an ALK1 antagonist, the VEGF antagonist, and an anti-angiogenesis agent.

The terms “VEGF” and “VEGF-A” are used interchangeably to refer to the 165-amino acid vascular endothelial cell growth factor and related 121-, 145-, 183-, 189-, and 206-amino acid vascular endothelial cell growth factors, as described by Leung et al. Science, 246:1306 (1989), Houck et al. Mol Endocrinol, 5:1806 (1991), and, Robinson & Stringer, J Cell Sci, 144(5):853-865 (2001), together with the naturally occurring allelic and processed forms thereof.

A “VEGF antagonist” refers to a molecule capable of neutralizing, blocking, inhibiting, abrogating, reducing or interfering with VEGF activities including its binding to one or more VEGF receptors. VEGF antagonists include anti-VEGF antibodies and antigen-binding fragments thereof, receptor molecules and derivatives which bind specifically to VEGF thereby sequestering its binding to one or more receptors, anti-VEGF receptor antibodies and VEGF receptor antagonists such as small molecule inhibitors of the VEGFR tyrosine kinases, and fusions proteins, e.g., VEGF-Trap (Regeneron), VEGF121-gelonin (Peregrine). VEGF antagonists also include antagonist variants of VEGF, antisense molecules directed to VEGF, RNA aptamers, and ribozymes against VEGF or VEGF receptors.

An “anti-VEGF antibody” is an antibody that binds to VEGF with sufficient affinity and specificity. The anti-VEGF antibody can be used as a therapeutic agent in targeting and interfering with diseases or conditions wherein the VEGF activity is involved. See, e.g., U.S. Pat. Nos. 6,582,959, 6,703,020; WO98/45332; WO 96/30046; WO94/10202, WO2005/044853; EP 0666868B1; U.S. Patent Applications 20030206899, 20030190317, 20030203409, 20050112126, 20050186208, and 20050112126; Popkov et al, Journal of Immunological Methods 288:149-164 (2004); and WO2005012359. An anti-VEGF antibody will usually not bind to other VEGF homologues such as VEGF-B or VEGF-C, nor other growth factors such as PlGF, PDGF or bFGF. The anti-VEGF antibody “Bevacizumab (BV)”, also known as “rhuMAb VEGF” or “Avastin®”, is a recombinant humanized anti-VEGF monoclonal antibody generated according to Presta et al. Cancer Res. 57:4593-4599 (1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies, including the anti-VEGF antibody fragment “ranibizumab”, also known as “Lucentis®”, are further described in U.S. Pat. No. 6,884,879 issued Feb. 26, 2005.

The term “anti-neoplastic composition” refers to a composition useful in treating cancer comprising at least one active therapeutic agent, e.g., “anti-cancer agent”. Examples of therapeutic agents (anti-cancer agents, also termed “anti-neoplastic agent” herein) include, but are not limited to, e.g., chemotherapeutic agents, growth inhibitory agents, cytotoxic agents, agents used in radiation therapy, anti-angiogenesis agents, apoptotic agents, anti-tubulin agents, toxins, and other-agents to treat cancer, e.g., anti-VEGF neutralizing antibody, VEGF antagonist, anti-HER-2, anti-CD20, an epidermal growth factor receptor (EGFR) antagonist (e.g., a tyrosine kinase inhibitor), HER1/EGFR inhibitor, erlotinib, a COX-2 inhibitor (e.g., celecoxib), interferons, cytokines, antagonists (e.g., neutralizing antibodies) that bind to one or more of the ErbB2, ErbB3, ErbB4, or VEGF receptor(s), inhibitors for receptor tyrosine kinases for platelet-derived growth factor (PDGF) and/or stem cell factor (SCF) (e.g., imatinib mesylate (Gleevec® Novartis)), TRAIL/Apo2L, and other bioactive and organic chemical agents, etc.

An “angiogenic factor or agent” is a growth factor which stimulates the development of blood vessels, e.g., promotes angiogenesis, endothelial cell growth, stability of blood vessels, and/or vasculogenesis, etc. For example, angiogenic factors, include, but are not limited to, e.g., VEGF and members of the VEGF family, PlGF, PDGF family, fibroblast growth factor family (FGFs), TIE ligands (Angiopoietins), ephrins, ANGPTL3, ALK-1, etc. It would also include factors that accelerate wound healing, such as growth hormone, insulin-like growth factor-I (IGF-I), VIGF, epidermal growth factor (EGF), CTGF and members of its family, and TGF-α and TGF-β. See, e.g., Klagsbrun and D'Amore, Annu. Rev. Physiol, 53:217-39 (1991); Streit and Detmar, Oncogene, 22:3172-3179 (2003); Ferrara & Alitalo, Nature Medicine 5(12): 1359-1364 (1999); Tonini et al., Oncogene, 22:6549-6556 (2003) (e.g., Table 1 listing angiogenic factors); and, Sato Int. J. Clin. Oncol., 8:200-206 (2003).

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. For example, an anti-angiogenesis agent is an antibody or other antagonist to an angiogenic agent as defined above, e.g., antibodies to VEGF, antibodies to VEGF receptors, small molecules that block VEGF receptor signaling (e.g., PTK787/ZK2284, SU6668, SUTENT®/SU 11248 (sunitinib malate), AMG706, or those described in, e.g., international patent application WO 2004/113304). Anti-angiogenesis agents also include native angiogenesis inhibitors, e.g., angiostatin, endostatin, etc. See, e.g., Klagsbrun and D'Amore, Annu. Rev. Physiol, 53:217-39 (1991); Streit and Detmar, Oncogene, 22:3172-3179 (2003) (e.g., Table 3 listing anti-angiogenic therapy in malignant melanoma); Ferrara & Alitalo, Nat Med 5(12): 1359-1364 (1999); Tonini et al, Oncogene, 22:6549-6556 (2003) (e.g., Table 2 listing antiangiogenic factors); and, Sato Int. J. Clin. Oncol, 8:200-206 (2003) (e.g., Table 1 lists Anti-angiogenesis agents used in clinical trials).

In certain aspects of the disclosure, other therapeutic agents useful for combination tumor therapy with an ALK1 antagonist include other cancer therapies: e.g., surgery, cytotoxic agents, radiological treatments involving irradiation or administration of radioactive substances, chemotherapeutic agents, anti-hormonal agents, growth inhibitory agents, anti-neoplastic compositions, and treatment with anti-cancer agents listed herein and known in the art, or combinations thereof.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also suitable for administration in combination with an ALK1 antagonist as described herein are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY1 17018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVIS OR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROC AL® etidronate, NE-58095, ZOMET A® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

A “growth inhibitory agent” when used herein refers to a compound or composition which inhibits growth of a cell either in vitro or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

In certain embodiments, the subject methods of the disclosure can be used alone. Alternatively, the subject methods may be used in combination with other conventional anti-cancer therapeutic approaches directed to treatment or prevention of proliferative disorders (e.g., tumor). For example, such methods can be used in prophylactic cancer prevention, prevention of cancer recurrence and metastases after surgery, and as an adjuvant of other conventional cancer therapy. The present disclosure recognizes that the effectiveness of conventional cancer therapies (e.g., chemotherapy, radiation therapy, phototherapy, immunotherapy, and surgery) can be enhanced through the use of a subject polypeptide therapeutic agent.

A wide array of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When an ALK1 antagonist described herein is administered in combination with another conventional anti-neoplastic agent, either concomitantly or sequentially, the ALK1 antagonist may enhance the therapeutic effect of the anti-neoplastic agent or overcome cellular resistance to the anti-neoplastic agent. This allows decrease of dosage of the anti-neoplastic agent, thereby reducing undesirable side effects, or restoring the effectiveness of an anti-neoplastic agent in resistant cells.

According to the present disclosure, the ALK1 antagonists described herein may be used in combination with other compositions and procedures for the treatment of diseases. For example, a tumor may be treated conventionally with surgery, radiation or chemotherapy combined with an ALK1 antagonist described herein, and/or the ALK1 antagonist may be subsequently administered to the patient to extend the dormancy of micrometastases and to stabilize any residual primary tumor.

ALK1 antagonists as described herein can also be given prophylactically to individuals identified to respond to ALK1 antagonists and known to be at an elevated risk for developing new or re-current cancers. Accordingly, some aspects of the disclosure encompasses methods for prophylactic prevention of ALK1 antagonist-responsive cancer in a subject, comprising administrating to the subject an effective amount of an ALK1 antagonist.

### 4. ALK1 Antagonists

ALK1 antagonists include agents that inhibit, interrupt, or prevent ALK1 signaling. In some embodiments, an ALK1 antagonist is a protein or polypeptide, while in other embodiments, an ALK1 antagonist is a nucleic acid or a small molecule. Exemplary ALK1 antagonists include soluble proteins or peptides comprising a ligand binding portion of the extracellular domain (ECD) of ALK1 (“ALK1 ECD polypeptides”), fusion proteins of ALK1-ECD polypeptides, e.g., ALK1-Fc proteins, in which an ALK1-ECD is fused to the Fc portion of an immunoglobulin, antibodies or antibody fragments specifically binding ALK1 or an ALK1 ligand, soluble endoglin ECD polypeptides, endoglin ECD fusion proteins, e.g., endoglin-ECD-Fc fusion proteins, and BMP9 and BMP10 pro-peptides. While not wishing to be bound to any particular mechanism of action, it is believed that such ALK1-antagonists bind to ALK1 or ALK1 ligands and inhibit the ability of ALK1 ligands to interact with ALK1. ALK1 antagonists also include small molecules that specifically bind to ALK1 or an ALK1 ligand and inhibit ALK1 signaling. In some preferred embodiments, the ALK1 antagonist comprises an ALK1-Fc fusion protein, e.g., a protein in which an ALK1 protein, or a fragment thereof, for example, an ALK1-ECD fragment, is fused to the Fc portion, or a fragment thereof, of an immunoglobulin.

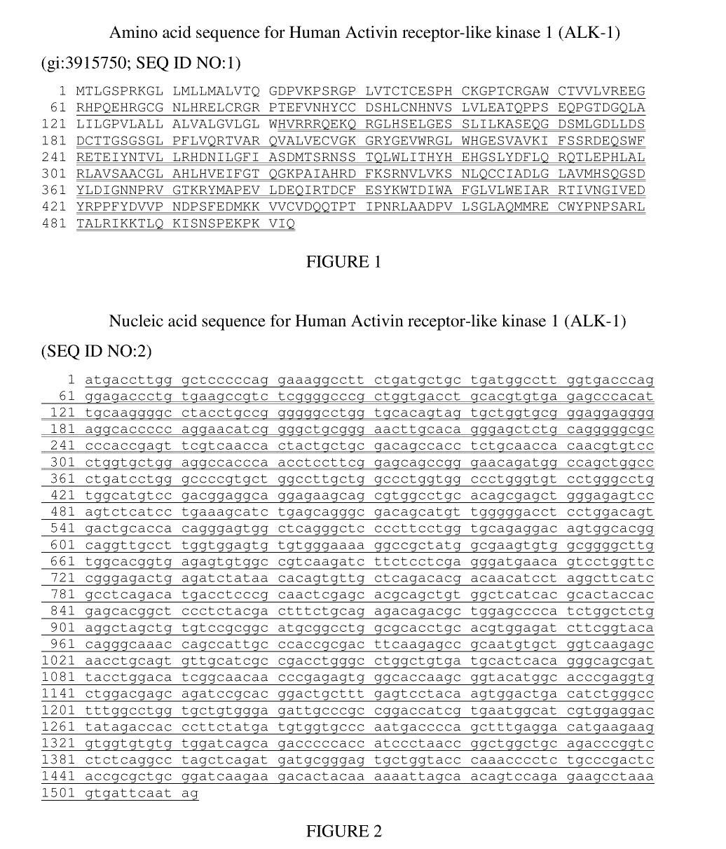

In preferred embodiments, the ALK1 antagonists, ALK1 ligands, ALK1 agonists, and other proteins described or referred to herein are the human forms, and the ALK1 antagonists referred to inhibit or disrupt human ALK1 signaling, unless otherwise specified. Sequences of ALK1 and ALK1 ligands, e.g., BMP9 and BMP10, and endoglin are known to those of skill in the art. Representative Genbank references for ALK1 ligands proteins are as follows: human BMP9: Q9UK05; human BMP10: 095393. The entire contents of these GenBank entries are incorporated herein by reference. Exemplary human BMP9, BMP10, endoglin, and ALK1 sequences are provided below. Additional representative ALK1 sequences are set forth in FIGS. 1-3. Sequences of ALK1, endoglin, and ALK1 ligands from other mammalian species will be apparent to those of skill in the art. It will also be apparent to the skilled artisan that the sequences provided herein are exemplary and serve to illustrate some of the embodiments described herein, but that the disclosure is not limited in this respect.

BMP9:
(SEQ ID NO: 10)
>gi|13124266|sp|Q9UK05.1|GDF2_HUMAN RecName:
Full = Growth/differentiation factor 2; Short = GDF-2; AltName: Full =
Bone morphogenetic protein 9; Short = BMP-9; Flags: Precursor
MCPGALWVALPLLSLLAGSLQGKPLQSWGRGSAGGNAHSPLGVPGGGLPEHTFNLKMFLENVKVDFLRS
LNLSGVPSQDKTRVEPPQYMIDLYNRYTSDKSTTPASNIVRSFSMEDAISITATEDFPFQKHILLFNIS
IPRHEQITRAELRLYVSCQNHVDPSHDLKGSVVIYDVLDGTDAWDSATETKTFLVSQDIQDEGWETLEV
SSAVKRWVRSDSTKSKNKLEVTVESHRKGCDTLDISVPPGSRNLPFFVVFSNDHSSGTKETRLELREMI
SHEQESVLKKLSKDGSTEAGESSHEEDTDGHVAAGSTLARRKRSAGAGSHCQKTSLRVNFEDIGWDSWI
IAPKEYEAYECKGGCFFPLADDVTPTKHAIVQTLVHLKFPTKVGKACCVPTKLSPISVLYKDDMGVPTL
KYHYEGMSVAECGCR (underline indicates the sequence of the mature BMP9
peptide)
BMP10:
(SEQ ID NO: 11)
>gi|13123977|sp|095393.1|BMP10_HUMAN RecName: Full = Bone morphogenetic
protein 10; Short = BMP-10; Flags: Precursor
MGSLVLTLCALFCLAAYLVSGSPIMNLEQSPLEEDMSLFGDVFSEQDGVDFNTLLQSMKDEFLKTLNLS
DIPTQDSAKVDPPEYMLELYNKFATDRTSMPSANIIRSFKNEDLFSQPVSFNGLRKYPLLFNVSIPHHE
EVIMAELRLYTLVQRDRMIYDGVDRKITIFEVLESKGDNEGERNMLVLVSGEIYGTNSEWETFDVTDAI
RRWQKSGSSTHQLEVHIESKHDEAEDASSGRLEIDTSAQNKHNPLLIVFSDDQSSDKERKEELNEMISH
EQLPELDNLGLDSFSSGPGEEALLQMRSNIIYDSTARIRRNAKGNYCKRTPLYIDFKEIGWDSWIIAPP
GYEAYECRGVCNYPLAEHLTPTKHAIIQALVHLKNSQKASKACCVPTKLEPISILYLDKGVVTYKFKYE
GMAVSECGCR (underline indicates the sequence of the mature BMP10
peptide)
ALK1
(SEQ ID NO: 12)
>gi|116734712|ref|NP_000011.2| serine/threonine-protein kinase
receptor R3 precursor [Homo sapiens]
MTLGSPRKGLLMLLMALVTQGDPVKPSRGPLVTCTCESPHCKGPTCRGAWCTVVLVREEGRHPQEHRGC
GNLHRELCRGRPTEFVNHYCCDSHLCNHNVSLVLEATQPPSEQPGTDGQLALILGPVLALLALVALGVL
GLWHVRRRQEKQRGLHSELGESSLILKASEQGDSMLGDLLDSDCTTGSGSGLPFLVQRTVARQVALVEC
VGKGRYGEVWRGLWHGESVAVKIFSSRDEQSWFRETEIYNTVLLRHDNILGFIASDMTSRNSSTQLWLI
THYHEHGSLYDFLQRQTLEPHLALRLAVSAACGLAHLHVEIFGTQGKPAIAHRDFKSRNVLVKSNLQCC
IADLGLAVMHSQGSDYLDIGNNPRVGTKRYMAPEVLDEQIRTDCFESYKWTDIWAFGLVLWEIARRTIV
NGIVEDYRPPFYDVVPNDPSFEDMKKVVCVDQQTPTIPNRLAADPVLSGLAQMMRECWYPNPSARLTAL
RIKKTLQKISNSPEKPKVIQ
Endoglin, isoform 1 (ENG)
(SEQ ID NO: 13)
>gi|168693647|ref|NP_001108225.1| endoglin isoform 1 precursor
[Homo sapiens]
MDRGTLPLAVALLLASCSLSPTSLAETVHCDLQPVGPERGEVTYTTSQVSKGCVAQAPNAILEVHVLFL
EFPTGPSQLELTLQASKQNGTWPREVLLVLSVNSSVFLHLQALGIPLHLAYNSSLVTFQEPPGVNTTEL
PSFPKTQILEWAAERGPITSAAELNDPQSILLRLGQAQGSLSFCMLEASQDMGRTLEWRPRTPALVRGC
HLEGVAGHKEAHILRVLPGHSAGPRTVTVKVELSCAPGDLDAVLILQGPPYVSWLIDANHNMQIWTTGE
YSFKIFPEKNIRGFKLPDTPQGLLGEARMLNASIVASFVELPLASIVSLHASSCGGRLQTSPAPIQTTP
PKDTCSPELLMSLIQTKCADDAMTLVLKKELVAHLKCTITGLTFWDPSCEAEDRGDKFVLRSAYSSCGM
QVSASMISNEAVVNILSSSSPQRKKVHCLNMDSLSFQLGLYLSPHFLQASNTIEPGQQSFVQVRVSPSV
SEFLLQLDSCHLDLGPEGGTVELIQGRAAKGNCVSLLSPSPEGDPRFSFLLHFYTVPIPKTGTLSCTVA
LRPKTGSQDQEVHRTVFMRLNIISPDLSGCTSKGLVLPAVLGITFGAFLIGALLTAALWYIYSHTRSPS
KREPVVAVAAPASSESSSTNHSIGSTQSTPCSTSSMA
Endoglin, isoform 2 (ENG)
(SEQ ID NO: 14)
>gi|4557555|ref|NP_000109.1| endoglin isoform 2 precursor
[Homo sapiens]
MDRGTLPLAVALLLASCSLSPTSLAETVHCDLQPVGPERGEVTYTTSQVSKGCVAQAPNAILEVHVLFL
EFPTGPSQLELTLQASKQNGTWPREVLLVLSVNSSVFLHLQALGIPLHLAYNSSLVTFQEPPGVNTTEL
PSFPKTQILEWAAERGPITSAAELNDPQSILLRLGQAQGSLSFCMLEASQDMGRTLEWRPRTPALVRGC
HLEGVAGHKEAHILRVLPGHSAGPRTVTVKVELSCAPGDLDAVLILQGPPYVSWLIDANHNMQIWTTGE
YSFKIFPEKNIRGFKLPDTPQGLLGEARMLNASIVASFVELPLASIVSLHASSCGGRLQTSPAPIQTTP
PKDTCSPELLMSLIQTKCADDAMTLVLKKELVAHLKCTITGLTFWDPSCEAEDRGDKFVLRSAYSSCGM
QVSASMISNEAVVNILSSSSPQRKKVHCLNMDSLSFQLGLYLSPHFLQASNTIEPGQQSFVQVRVSPSV
SEFLLQLDSCHLDLGPEGGTVELIQGRAAKGNCVSLLSPSPEGDPRFSFLLHFYTVPIPKTGTLSCTVA
LRPKTGSQDQEVHRTVFMRLNIISPDLSGCTSKGLVLPAVLGITFGAFLIGALLTAALWYIYSHTREYP
RPPQ

In some embodiments, the terms “BMP9” and “BMP10” refer to a gene product, e.g., a nucleic acid, protein, or peptide encoded by a BMP9 or BMP10 gene, respectively. In some embodiments, the terms refer to a BMP9 or BMP10 precursor, or to any naturally occurring cleavage product thereof. In some embodiments, the terms refer to a mature BMP9 or BMP10 polypeptide, for example, a naturally occurring mature BMP9 or BMP10 polypeptide.

While some ALK1 antagonists useful in the methods provided by this disclosure are described in detail herein, additional ALK1 antagonists useful according to aspects of this disclosure will be apparent to those of skill in the art based on the description provided herein. For additional ALK1 antagonists that may be used in the methods and kits provided herein, see, e.g., Additional ALK1-Fc fusion proteins that are useful as ALK1 antagonists in the methods of this invention are known to those of skill in the art. See, e.g., Cunha et al., J Exp Med 2010 207(1):85-100; PCT Application Publication WO/2009/134428; WO/2008/057461; WO/2009/139891; and WO/2008/151078; and U.S. Pat. No. 7,741,284; the entire contents of each of which are incorporated herein by reference. It will be understood that the disclosure is not limited in this respect.

A. ALK1-ECD Polypeptides

Naturally occurring ALK1 proteins are transmembrane proteins, with a portion of the protein positioned outside the cell (the extracelluar portion or extracellular domain) and a portion of the protein positioned inside the cell (the intracellular portion or intracellular domain). Aspects of the present disclosure encompass polypeptides comprising a portion of the extracellular domain of ALK1 and their use as ALK1 antagonists in the methods described herein.

In certain embodiments, the disclosure provides “ALK1 ECD polypeptides” that function as ALK1 antagonists. The term “ALK1 ECD polypeptide” refers to a polypeptide consisting of or comprising an amino acid sequence of an extracellular domain of a naturally occurring ALK1 polypeptide, either including or excluding any signal sequence and sequence N-terminal to the signal sequence, or an amino acid sequence that is at least 33 percent identical to an extracellular domain of a naturally occurring ALK1 polypeptide, and, optionally, at least 60%, at least 70%, at least 80%, at least 85%, at least 90%, at least 95%, at least 96%, at least 97%, at least 98%, at least 99% or 100% identical to the sequence of an extracellular domain of a naturally occurring ALK1 polypeptide, as exemplified by the cysteine knot region of amino acids 34-95 of SEQ ID NO:1 or the cysteine knot plus additional amino acids at the N- and C-termini of the extracellular domain, such as amino acids 22-118 of SEQ ID NO. 1. In some embodiments, a polypeptide comprising the cysteine knot region of amino acids 34-95 may be employed as an ALK1 antagonist.

Likewise, an ALK1 ECD polypeptide may comprise a polypeptide that is encoded by nucleotides 100-285 of SEQ ID NO:2, or silent variants thereof or nucleic acids that hybridize to the complement thereof under stringent hybridization conditions (generally, such conditions are known in the art but may, for example, involve hybridization in 50% v/v formamide, 5×SSC, 2% w/v blocking agent, 0.1% N-lauroylsarcosine, 0.3% SDS at 65 C.° overnight and washing in, for example, 5×SSC at about 65 C.°). Additionally, an ALK1 ECD polypeptide may comprise a polypeptide that is encoded by nucleotides 64-384 of SEQ ID NO:2, or silent variants thereof or nucleic acids that hybridize to the complement thereof under stringent hybridization conditions (generally, such conditions are known in the art but may, for example, involve hybridization in 50% v/v formamide, 5×SSC, 2% w/v blocking agent, 0.1% N-lauroylsarcosine, 0.3% SDS at 65 C.° overnight and washing in, for example, 5×SSC at about 65 C.°). The term “ALK1 ECD polypeptide” accordingly encompasses isolated extracellular portions of ALK1 polypeptides, variants thereof (including variants that comprise, for example, no more than 2, 3, 4, 5 or 10 amino acid substitutions, additions or deletions in the sequence corresponding to amino acids 22-118 of SEQ ID NO:1 and including variants that comprise no more than 2, 3, 4, 5, or 10 amino acid substitutions, additions or deletions in the sequence corresponding to amino acids 34-95 of SEQ ID NO:1), fragments thereof and fusion proteins comprising any of the preceding, but in each case preferably any of the foregoing ALK1 ECD polypeptides will retain substantial affinity for one or more of GDF5, GDF6, GDF7, BMP9 or BMP10. The term “ALK1 ECD polypeptide” is explicitly intended to exclude any full-length, naturally occurring ALK1 polypeptide. Generally, an ALK1 ECD polypeptide will be designed to be soluble in aqueous solutions at biologically relevant temperatures, pH levels and osmolarity.

As described above, the disclosure provides ALK1 ECD polypeptides sharing a specified degree of sequence identity or similarity to a naturally occurring ALK1 polypeptide. To determine the percent identity of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues at corresponding amino acid positions are then compared. When a position in the first sequence is occupied by the same amino acid residue as the corresponding position in the second sequence, then the molecules are identical at that position (as used herein amino acid “identity” is equivalent to amino acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

In some embodiments, the percent identity between two amino acid sequences is determined using the Needleman and Wunsch (J Mol. Biol. (48):444-453 (1970)) algorithm which has been incorporated into the GAP program in the GCG software package (available at http://www.gcg.com). In a specific embodiment, the following parameters are used in the GAP program: either a Blosum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (Devereux, J., et al., Nucleic Acids Res. 12(1):387 (1984)) (available at http://www.gcg.com). Exemplary parameters include using a NWSgapdna.CMP matrix and a gap weight of 40, 50, 60, 70, or 80 and a length weight of 1, 2, 3, 4, 5, or 6. Unless otherwise specified, percent identity between two amino acid sequences is to be determined using the GAP program using a Blosum 62 matrix, a GAP weight of 10 and a length weight of 3, and if such algorithm cannot compute the desired percent identity, a suitable alternative disclosed herein should be selected.

In some embodiments, the percent identity between two amino acid sequences is determined using the algorithm of E. Myers and W. Miller (CABIOS, 4:11-17 (1989)) which has been incorporated into the ALIGN program (version 2.0), using a PAM120 weight residue table, a gap length penalty of 12 and a gap penalty of 4.

In some embodiments, the best overall alignment between two amino acid sequences can be determined using the FASTDB computer program based on the algorithm of Brutlag et al. (Comp. App. Biosci., 6:237-245 (1990)). In a sequence alignment the query and subject sequences are both amino acid sequences. The result of said global sequence alignment is presented in terms of percent identity. In one embodiment, amino acid sequence identity is performed using the FASTDB computer program based on the algorithm of Brutlag et al. (Comp. App. Biosci., 6:237-245 (1990)). In a specific embodiment, parameters employed to calculate percent identity and similarity of an amino acid alignment comprise: Matrix=PAM 150, k-tuple=2, Mismatch Penalty=1, Joining Penalty=20, Randomization Group Length=0, Cutoff Score=1, Gap Penalty=5 and Gap Size Penalty=0.05.

In certain embodiments, ALK1 ECD polypeptides comprise an extracellular portion of a naturally occurring ALK1 protein such as a sequence of SEQ ID NO:1, and preferably a ligand binding portion of the ALK1 extracellular domain. In certain embodiments, a soluble ALK1 polypeptide comprises an amino acid sequence that is at least 60%, at least 70%, at least 80%, at least 85%, at least 90%, at least 95%, at least 96%, at least 97%, at least 98% or at least 99% identical to an amino acid sequence of amino acids 22-118 of the SEQ ID NO:1. In certain embodiments, a truncated extracellular ALK1 polypeptide comprises at least 30, 40 or 50 consecutive amino acids of an amino acid sequence of an extracellular portion of SEQ ID NO:1. In some embodiments, a soluble ALK1 polypeptide useful as an ALK1 antagonist in the methods described herein comprises an amino acid sequence that is at that is at least 60%, at least 70%, at least 80%, at least 85%, at least 90%, at least 95%, at least 96%, at least 97%, at least 98% or at least 99% identical to an amino acid sequence comprising 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or more than 80 consecutive amino acids within the sequence of amino acids 22-118 of SEQ ID NO:1. In some embodiments, a soluble ALK1 polypeptide useful as an ALK1 antagonist in the methods described herein meets the similarity requirements set forth above and comprises a cystein knot structure.

In preferred embodiments, an ALK1 ECD polypeptide binds to an ALK1 ligand, e.g., to BMP9 and/or BMP10. Optionally the ALK1 polypeptide does not show substantial binding to TGF-131 or TGF-133. Binding may be assessed using purified proteins in solution or in a surface plasmon resonance system, such as a Biacore™ system. Preferred soluble ALK1 polypeptides will exhibit an anti-angiogenic activity. Bioassays for angiogenesis inhibitory activity include the chick chorioallantoic membrane (CAM) assay, the mouse corneal micropocket assay, an assay for measuring the effect of administering isolated or synthesized proteins on implanted tumors. The CAM assay is described by O'Reilly, et al. in “Angiogenic Regulation of Metastatic Growth” Cell, vol. 79 (2), Oct. 1, 1994, pp. 315-328. Briefly, 3 day old chicken embryos with intact yolks are separated from the egg and placed in a petri dish. After 3 days of incubation, a methylcellulose disc containing the protein to be tested is applied to the CAM of individual embryos. After 48 hours of incubation, the embryos and CAMs are observed to determine whether endothelial growth has been inhibited. The mouse corneal micropocket assay involves implanting a growth factor-containing pellet, along with another pellet containing the suspected endothelial growth inhibitor, in the cornea of a mouse and observing the pattern of capillaries that are elaborated in the cornea. Other assays are described in the Examples.

ALK1 ECD polypeptides may be produced by removing the cytoplasmic tail and the transmembrane region of an ALK1 polypeptide. Alternatively, the transmembrane domain may be inactivated by deletion, or by substitution of the normally hydrophobic amino acid residues which comprise a transmembrane domain with hydrophilic ones. In either case, a substantially hydrophilic hydropathy profile is created which will reduce lipid affinity and improve aqueous solubility. Deletion of the transmembrane domain is preferred over substitution with hydrophilic amino acid residues because it avoids introducing potentially immunogenic epitopes.

ALK1 ECD polypeptides may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native ALK1 signal sequence may be used to effect extrusion from the cell. Possible leader sequences include native, tPa and honeybee mellitin leaders (SEQ ID NOs. 7-9, respectively). Processing of signal peptides may vary depending on the leader sequence chosen, the cell type used and culture conditions, among other variables, and therefore actual N-terminal start sites for mature ALK1 ECD polypeptides may shift by 1-5 amino acids in either the N-terminal or C-terminal direction.

In certain embodiments, the present disclosure contemplates specific mutations of the ALK1 polypeptides so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (or asparagines-X-serine) (where “X” is any amino acid) which is specifically recognized by appropriate cellular glycosylation enzymes. The alteration may also be made by the addition of, or substitution by, one or more serine or threonine residues to the sequence of the wild-type ALK1 polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on an ALK1 polypeptide is by chemical or enzymatic coupling of glycosides to the ALK1 polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on an ALK1 polypeptide may be accomplished chemically and/or enzymatically. Chemical deglycosylation may involve, for example, exposure of the ALK1 polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on ALK1 polypeptides can be achieved by the use of a variety of endo- and exo-glycosidases as described by Thotakura et al. (1987) Meth. Enzymol. 138:350. The sequence of an ALK1 polypeptide may be adjusted, as appropriate, depending on the type of expression system used, as mammalian, yeast, insect and plant cells may all introduce differing glycosylation patterns that can be affected by the amino acid sequence of the peptide. In general, ALK1 proteins for use in humans will be expressed in a mammalian cell line that provides proper glycosylation, such as HEK293 or CHO cell lines, although other mammalian expression cell lines, yeast cell lines with engineered glycosylation enzymes and insect cells are expected to be useful as well.

This disclosure further contemplates the use of mutants, particularly sets of combinatorial mutants of an ALK1 polypeptide, as well as truncation mutants, as ALK1 antagonists in the methods described herein. Pools of combinatorial mutants are especially useful for identifying functional variant sequences. The purpose of screening such combinatorial libraries may be to generate, for example, ALK1 polypeptide variants which can act as ALK1 antagonists. A variety of suitable screening assays are provided in U.S. Patent Application Publication US2008/0175844 A1 and U.S. Pat. No. 8,158,584, the entire contents of each of which are incorporated herein by reference, and such assays may be used to evaluate variants. Additional useful screening assays will be apparent to those of skill in the art and this disclosure is not limited in this respect.

In certain embodiments, the ALK1 ECD polypeptides useful as ALK1 antagonists may further comprise post-translational modifications in addition to any that are naturally present in the ALK1 polypeptides. Such modifications include, but are not limited to, acetylation, carboxylation, glycosylation, phosphorylation, lipidation, and acylation. As a result, the modified ALK1 ECD polypeptides may contain non-amino acid elements, such as polyethylene glycols, lipids, poly- or mono-saccharide, and phosphates. Effects of such non-amino acid elements on the functionality of an ALK1 ECD polypeptide may be tested as described herein for other ALK1 ECD polypeptide variants. When an ALK1 ECD polypeptide is produced in cells by cleaving a nascent form of the ALK1 polypeptide, post-translational processing may also be important for correct folding and/or function of the protein. Different cells (such as CHO, HeLa, MDCK, 293, WI38, NIH-3T3 or HEK293) have specific cellular machinery and characteristic mechanisms for such post-translational activities and may be chosen to ensure the correct modification and processing of the ALK1 polypeptides.

ALK 1 antagonistic polypeptides comprising a portion of the extracellular domain of ALK1 (“ALK1 ECD polypeptides”) may be used to inhibit angiogenesis in vivo, including VEGF-independent angiogenesis and angiogenesis that is mediated by multiple angiogenic factors, including VEGF, FGF and PDGF. In part, the disclosure provides the identity of physiological, high affinity ligands for ALK1, including BMP9 and BMP10 and demonstrates that ALK1 ECD polypeptides inhibit ALK1 ligand-mediated angiogenesis. The data presented herein demonstrate that an ALK1 ECD polypeptide can exert an anti-angiogenic, ALK1-antagonistic effect even in the case where the ALK1 ECD polypeptide does not exhibit meaningful binding to TGF-β1.

While an ALK1 ECD polypeptide inhibits all of the ligands that it binds to tightly, including, for example, BMP9 and BMP10, it does not affect signaling mediated through ligands that it binds to weakly, such as TGF-β. Accordingly, an ALK1 ECD polypeptide inhibits BMP9 and BMP10 signaling through all receptors (including receptors other than ALK1), but does not inhibit TGF-β signaling through any receptor, even ALK1. This is in contrast to ALK1 antagonists provided herein that directly bind to ALK1. For example, a pan-neutralizing antibody against ALK1 blocks BMP9, BMP10, and TGF-β signaling through ALK1, but it would not block BMP9 and TGF-β signaling through another receptor.

B. ALK1-ECD Fusion Proteins

In certain embodiments, functional variants or modified forms of the ALK1 ECD polypeptides useful as ALK1 antagonists in the presently disclosed methods include fusion proteins having at least a portion of the ALK1 ECD polypeptides and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners.

As another example, a fusion domain may be selected so as to facilitate detection of the ALK1 ECD polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus hemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain preferred embodiments, an ALK1 ECD polypeptide is fused with a domain that stabilizes the ALK1 polypeptide in vivo (a “stabilizer” domain). By “stabilizing” is meant anything that increases serum half-life, regardless of whether this is because of decreased destruction, decreased clearance by the kidney, or other pharmacokinetic effect.

Fusions with the Fc portion of an immunoglobulin are known to confer desirable pharmacokinetic properties on a wide range of proteins. Likewise, fusions to human serum albumin can confer desirable properties. Other types of fusion domains that may be selected include multimerizing (e.g., dimerizing, tetramerizing) domains and functional domains. As a specific example, the present disclosure provides an ALK1 antagonist comprising a fusion protein comprising a soluble extracellular domain of ALK1 fused to an Fc domain (e.g., SEQ ID NO: 6).

THTCPPCPAPELLGGPSVFLFPPKPKDTLMISRTPEVTCVVVD (A) VSHEDPEVKFN
WYVDGVEVHNAKTKPREEQYNSTYRVVSVLTVLHQDWLNGKEYKCK (A) VSNKALPV
PIEKTISKAKGQPREPQVYTLPPSREEMTKNQVSLTCLVKGFYPSDIAVEWESNGQP
ENNYKTTPPVLDSDGPFFLYSKLTVDKSRWQQGNVFSCSVMHEALHN (A) HYTQKSL
SLSPGK*

In some embodiments, the Fc domain has one or more mutations at residues such as Asp-265, lysine 322, and Asn-434 (underlined). In certain embodiments, the mutant Fc domain having one or more of these mutations (e.g., Asp-265 mutation) has reduced ability of binding to the Fey receptor relative to a wildtype Fc domain. In other cases, the mutant Fc domain having one or more of these mutations (e.g., Asn-434 mutation) has increased ability of binding to the MHC class I-related Fc-receptor (FcRN) relative to a wildtype Fc domain.

It is understood that different elements of the fusion proteins provided herein may be arranged in any manner that is consistent with the desired functionality as ALK1 antagonists. For example, an ALK1 ECD polypeptide may be placed C-terminal to a heterologous domain, or, alternatively, a heterologous domain may be placed C-terminal to an ALK1 ECD polypeptide. The ALK1 ECD polypeptide domain and the heterologous domain need not be adjacent in a fusion protein, and additional domains or amino acid sequences may be included C- or N-terminal to either domain or between the domains, e.g., as linkers.

As used herein, the term, “immunoglobulin Fc region” or simply “Fc” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain.

In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant region is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc γ or the homologous domains in any of IgA, IgD, IgE, or IgM.

Furthermore, it is contemplated that substitution or deletion of amino acids within the immunoglobulin heavy chain constant regions may be useful in the practice of the methods and compositions disclosed herein. One example would be to introduce amino acid substitutions in the upper CH2 region to create an Fc variant with reduced affinity for Fc receptors (Cole et al. (1997) J. Immunol. 159:3613).

In certain embodiments, the present disclosure makes available isolated and/or purified forms of the ALK1 ECD polypeptides, which are isolated from, or otherwise substantially free of (e.g., at least 80%, 90%, 95%, 96%, 97%, 98% or 99% free of), other proteins and/or other ALK1 ECD polypeptide species. ALK1 polypeptides will generally be produced by expression from recombinant nucleic acids.

In certain embodiments, the disclosure includes nucleic acids encoding soluble ALK1 polypeptides comprising the coding sequence for an extracellular portion of an ALK1 proteins. In further embodiments, this disclosure also pertains to a host cell comprising such nucleic acids. The host cell may be any prokaryotic or eukaryotic cell. For example, a polypeptide of the present disclosure may be expressed in bacterial cells such as E. coli, insect cells (e.g., using a baculovirus expression system), yeast, or mammalian cells. Other suitable host cells are known to those skilled in the art. Accordingly, some embodiments of the present disclosure further pertain to methods of producing the ALK1 ECD polypeptides. It has been established that an ALK1-Fc fusion protein set forth in SEQ ID NO:3 and expressed in CHO cells has potent anti-angiogenic activity.

C. Soluble Endoglin (ENG) Polypeptides

In some embodiments, soluble ENG polypeptides are used as ALK1 antagonists in the methods and kits provided herein. Naturally occurring ENG proteins are typically transmembrane proteins, with a portion of the protein positioned outside the cell (the extracelluar portion) and a portion of the protein positioned inside the cell (the intracellular portion). Aspects of the present disclosure encompass polypeptides comprising a portion of the extracellular domain (ECD) of ENG.

In certain embodiments, the disclosure provides ENG polypeptides as ALK1 antagonists. In some embodiments, the ENG polypeptide comprises an ENG-ECD polypeptide, for example, a full-length ENG-ECD as provided in SEQ ID NO: 15, or a truncated form of the ENG-ECD provided, for example, a polypeptide comprising at least 20, at least 30, at least 40, at least 50, at least 60, at least 70, at least 80, at least 100, at least 150, at least 200, at least 250, at least 400, or at least 500 contiguous amino acids of the ENG-ECD provided in SEQ ID NO: 15:

(SEQ ID NO: 15)
ETVHCDLQPVGPERGEVTYTTSQVSKGCVAQAPNAILEVHVLFLEFPTGPSQL
ELTLQASKQNGTWPREVLLVLSVNSSVFLHLQALGIPLHLAYNSSLVTFQEPP
GVNTTELPSFPKTQILEWAAERGPITSAAELNDPQSILLRLGQAQGSLSFCMLE
ASQDMGRTLEWRPRTPALVRGCHLEGVAGHKEAHILRVLPGHSAGPRTVTV
KVELSCAPGDLDAVLILQGPPYVSWLIDANHNMQIWTTGEYSFKIFPEKNIRG
FKLPDTPQGLLGEARMLNASIVASFVELPLASIVSLHASSCGGRLQTSPAPIQT
TPPKDTCSPELLMSLIQTKCADDAMTLVLKKELVAHLKCTITGLTFWDPSCEA
EDRGDKFVLRSAYSSCGMQVSASMISNEAVVNILSSSSPQRKKVHCLNMDSL
SFQLGLYLSPHFLQASNTIEPGQQSFVQVRVSPSVSEFLLQLDSCHLDLGPEGG
TVELIQGRAAKGNCVSLLSPSPEGDPRFSFLLHFYTVPIPKTGTLSCTVALRPK
TGSQDQEVHRTVFMRLNIISPDLSGCTSKG

An ALK1-antagonistic ENG polypeptides may include a polypeptide consisting of, or comprising, an amino acid sequence at least 90% identical, and optionally at least 95%, 96%, 97%, 98%, 99%, or 100% identical to a truncated ECD domain of a naturally occurring ENG polypeptide, whose C-terminus occurs at any of amino acids 333-378 of SEQ ID NO: 13 and which polypeptide does not include a sequence consisting of amino acids 379-430 of SEQ ID NO:13. Optionally, an ENG polypeptide does not include more than 5 consecutive amino acids, or more than 10, 20, 30, 40, 50, 52, 60, 70, 80, 90, 100, 150 or 200 or more consecutive amino acids from a sequence consisting of amino acids 379-586 of SEQ ID NO: 13 or from a sequence consisting of amino acids 379-581 of SEQ ID NO:13. The unprocessed ENG polypeptide may either include or exclude any signal sequence, as well as any sequence N-terminal to the signal sequence. As elaborated herein, the N-terminus of the mature (processed) ENG polypeptide may occur at any of amino acids 26-42 of SEQ ID NO: 13. Examples of additional ENG polypeptides useful as ALK1 antagonists are also described in U.S. Pat. No. 5,830,847, and in PCT Application PCT/US2012/034295, the entire contents of each of which are incorporated herein by reference.

Fc fusion proteins comprising shorter C-terminally truncated variants of ENG polypeptides display no appreciable binding to TGF-131 and TGF-133 but instead display higher affinity binding to BMP-9, with a markedly slower dissociation rate, compared to either ENG(26-437)-Fc or an Fc fusion protein comprising the full-length ENG ECD. Specifically, C-terminally truncated variants ending at amino acids 378, 359, and 346 of SEQ ID NO: 13 were all found to bind BMP-9 with substantially higher affinity (and to bind BMP-10 with undiminished affinity) compared to ENG(26-437) or ENG(26-586). However, binding to BMP-9 and BMP-10 was completely disrupted by more extensive C-terminal truncations to amino acids 332, 329, or 257. Thus, ENG polypeptides that terminate between amino acid 333 and amino acid 378 are all expected to be active, but constructs ending at, or between, amino acids 346 and 359 may be most active. Forms ending at, or between, amino acids 360 and 378 are predicted to trend toward the intermediate ligand binding affinity shown by ENG(26-378). Improvements in other key parameters are expected with certain constructs ending at, or between, amino acids 333 and 378 based on improvements in protein expression and elimination half-life observed with ENG(26-346)-Fc compared to fusion proteins comprising full-length ENG ECD (see Examples). Any of these truncated variant forms may be used as ALK1 antagonists according to aspects of this disclosure.

At the N-terminus, it is expected that an ENG polypeptide beginning at amino acid 26 (the initial glutamate), or before, of SEQ ID NO: 13 will retain ligand binding activity. As disclosed herein, an N-terminal truncation to amino acid 61 of SEQ ID NO: 1 abolishes ligand binding, as do more extensive N-terminal truncations. However, as also disclosed herein, consensus modeling of ENG primary sequences indicates that ordered secondary structure within the region defined by amino acids 26-60 of SEQ ID NO: 1 is limited to a four-residue beta strand predicted with high confidence at positions 42-45 of SEQ ID NO: 1 and a two-residue beta strand predicted with very low confidence at positions 28-29 of SEQ ID NO: 1. Thus, in some embodiments, an active ENG polypeptide will begin at (or before) amino acid 26, preferentially, or at any of amino acids 27-42 of SEQ ID NO: 13.

Taken together, an active portion of an ENG polypeptide may comprise amino acid sequences 26-333, 26-334, 26-335, 26-336, 26-337, 26-338, 26-339, 26-340, 26-341, 26-342, 26-343, 26-344, 26-345, or 26-346 of SEQ ID NO: 13, as well as variants of these sequences starting at any of amino acids 27-42 of SEQ ID NO: 13. Exemplary ENG polypeptides comprise amino acid sequences 26-346, 26-359, and 26-378 of SEQ ID NO: 13. Variants within these ranges are also contemplated, particularly those having at least 80%, 85%, 90%, 95%, or 99% identity to the corresponding portion of SEQ ID NO: 13. In some embodiments, an ENG polypeptide may not include the sequence consisting of amino acids 379-430 of SEQ ID NO:13.

ALK1-antagonistic ENG polypeptides useful according to some aspects of this disclosure may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native ENG signal sequence may be used to effect extrusion from the cell. Possible leader sequences include honeybee mellitin, TPA, and native leaders (SEQ ID NOs. 7-9, respectively). Processing of signal peptides may vary depending on the leader sequence chosen, the cell type used and culture conditions, among other variables, and therefore actual N-terminal start sites for mature ENG polypeptides may shift by 1, 2, 3, 4 or 5 amino acids in either the N-terminal or C-terminal direction. Examples of mature ENG-Fc fusion proteins include SEQ ID NOs: 16-19, as shown below with the ENG polypeptide portion underlined.

Human ENG(26-378)-hFc (truncated Fc)
(SEQ ID NO: 16)
ETVHCDLQPVGPERDEVTYTTSQVSKGCVAQAPNAILEVHVLFLEFPTGPSQLELTL
QASKQNGTWPREVLLVLSVNSSVFLHLQALGIPLHLAYNSSLVTFQEPPGVNTTELP
SFPKTQILEWAAERGPITSAAELNDPQSILLRLGQAQGSLSFCMLEASQDMGRTLEW
RPRTPALVRGCHLEGVAGHKEAHILRVLPGHSAGPRTVTVKVELSCAPGDLDAVLIL
QGPPYVSWLIDANHNMQIWTTGEYSFKIFPEKNIRGFKLPDTPQGLLGEARMLNASI
VASFVELPLASIVSLHASSCGGRLQTSPAPIQTTPPKDTCSPELLMSLIQTKCADDA
MTLVLKKELVATGGGTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNH
YTQKSLSLSPGK
Human ENG(26-359)-hFc
(SEQ ID NO: 17)
ETVHCDLQPVGPERDEVTYTTSQVSKGCVAQAPNAILEVHVLFLEFPTGPSQLELTL
QASKQNGTWPREVLLVLSVNSSVFLHLQALGIPLHLAYNSSLVTFQEPPGVNTTELP
SFPKTQILEWAAERGPITSAAELNDPQSILLRLGQAQGSLSFCMLEASQDMGRTLEW
RPRTPALVRGCHLEGVAGHKEAHILRVLPGHSAGPRTVTVKVELSCAPGDLDAVLIL
QGPPYVSWLIDANHNMQIWTTGEYSFKIFPEKNIRGFKLPDTPQGLLGEARMLNASI
VASFVELPLASIVSLHASSCGGRLQTSPAPIQTTPPKDTCSPELLMSLITGGGPKSC
DKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
Human ENG(26-359)-hFc (truncated Fc)
(SEQ ID NO: 18)
ETVHCDLQPVGPERDEVTYTTSQVSKGCVAQAPNAILEVHVLFLEFPTGPSQLELTL
QASKQNGTWPREVLLVLSVNSSVFLHLQALGIPLHLAYNSSLVTFQEPPGVNTTELP
SFPKTQILEWAAERGPITSAAELNDPQSILLRLGQAQGSLSFCMLEASQDMGRTLEW
RPRTPALVRGCHLEGVAGHKEAHILRVLPGHSAGPRTVTVKVELSCAPGDLDAVLIL
QGPPYVSWLIDANHNMQIWTTGEYSFKIFPEKNIRGFKLPDTPQGLLGEARMLNASI
VASFVELPLASIVSLHASSCGGRLQTSPAPIQTTPPKDTCSPELLMSLITGGGTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
Human ENG(26-346)-hFc (truncated Fc)
(SEQ ID NO: 19)
ETVHCDLQPVGPERDEVTYTTSQVSKGCVAQAPNAILEVHVLFLEFPTGPSQLELTL
QASKQNGTWPREVLLVLSVNSSVFLHLQALGIPLHLAYNSSLVTFQEPPGVNTTELP
SFPKTQILEWAAERGPITSAAELNDPQSILLRLGQAQGSLSFCMLEASQDMGRTLEW
RPRTPALVRGCHLEGVAGHKEAHILRVLPGHSAGPRTVTVKVELSCAPGDLDAVLIL
QGPPYVSWLIDANHNMQIWTTGEYSFKIFPEKNIRGFKLPDTPQGLLGEARMLNASI
VASFVELPLASIVSLHASSCGGRLQTSPAPIQTTPPTGGGTHTCPPCPAPELLGGPS
VFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK

In certain embodiments, the present disclosure contemplates specific mutations of the ENG polypeptides so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (or asparagines-X-serine) (where “X” is any amino acid) which is specifically recognized by appropriate cellular glycosylation enzymes. The alteration may also be made by the addition of, or substitution by, one or more serine or threonine residues to the sequence of the wild-type ENG polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on an ENG polypeptide is by chemical or enzymatic coupling of glycosides to the ENG polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on an ENG polypeptide may be accomplished chemically and/or enzymatically. Chemical deglycosylation may involve, for example, exposure of the ENG polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on ENG polypeptides can be achieved by the use of a variety of endo- and exo-glycosidases as described by Thotakura et al. (1987) Meth. Enzymol. 138:350. The sequence of an ENG polypeptide may be adjusted, as appropriate, depending on the type of expression system used, as mammalian, yeast, insect and plant cells may all introduce differing glycosylation patterns that can be affected by the amino acid sequence of the peptide. In general, ENG polypeptides for use in humans will be expressed in a mammalian cell line that provides proper glycosylation, such as HEK293 or CHO cell lines, although other mammalian expression cell lines, yeast cell lines with engineered glycosylation enzymes, and insect cells are expected to be useful as well.

D. Antibodies

Another aspect of the disclosure pertains to an ALK1-antagonistic antibodies. These include antibodies that disrupt the binding of ALK1 to an ALK1 ligand, e.g., to BMP9 and/or BMP10. In some embodiments, the ALK1-antagonistic antibody is an antibody reactive with an extracellular portion of an ALK1 polypeptide, preferably an antibody that specifically binds to an ALK1 polypeptide ECD as described herein. In a preferred embodiment, such antibody interferes with ALK1 binding to a ligand such BMP9 and/or BMP10. In other embodiments, the ALK1-antagonistic antibody is an antibody reactive with an ALK1 ligand, e.g., with BMP9 and/or BMP10. In a preferred embodiment, such antibody interferes with ALK1 binding to the ligand. It will be understood that an ALK1-antagonistic antibody, to be functional in vivo, should bind to the mature, processed form of the relevant protein. e.g., the ALK1 protein or the respective ligand. Preferred antibodies are those that exhibit an anti-angiogenic activity in a bioassay, such as a CAM assay or corneal micropocket assay.

The term “antibody” as used herein is intended to include whole antibodies, e.g., of any isotype (IgG, IgA, IgM, IgE, etc.), and includes fragments or domains of immunoglobulins which are reactive with a selected antigen. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant antibodies. The term “recombinant antibody”, means an antibody, or antigen binding domain of an immunoglobulin, expressed from a nucleic acid that has been constructed using the techniques of molecular biology, such as a humanized antibody or a fully human antibody developed from a single chain antibody. Single domain and single chain antibodies are also included within the term “recombinant antibody”.

Antibodies may be generated by any of the various methods known in the art, including administration of antigen to an animal, administration of antigen to an animal that carries human immunoglobulin genes, or screening with an antigen against a library of antibodies (often single chain antibodies or antibody domains). Once antigen binding activity is detected, the relevant portions of the protein may be grafted into other antibody frameworks, including full-length IgG frameworks. For example, by using immunogens derived from an ALK1 polypeptide or an ALK1 ligand, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a ALK1 polypeptide or an antigenic fragment which is capable of eliciting an antibody response, or a fusion protein). Techniques for conferring immunogenicity on a protein or peptide include conjugation to carriers or other techniques well known in the art. An immunogenic portion (preferably an extracellular portion) of an ALK1 polypeptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of an ALK1 polypeptide or an ALK1 ligand, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian ALK1 polypeptide of the present disclosure and monoclonal antibodies isolated from a culture comprising such hybridoma cells.

The term antibody as used herein is intended to include fragments thereof which are also specifically reactive with one of the subject ALK1 polypeptides or ALK1 ligands. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present disclosure is further intended to include bispecific, single-chain, and chimeric and humanized molecules having affinity for an ALK1 polypeptide or ALK1 ligand conferred by at least one CDR region of the antibody. In preferred embodiments, the antibody further comprises a label attached thereto and is able to be detected, (e.g., the label can be a radioisotope, fluorescent compound, enzyme or enzyme co-factor).

In certain preferred embodiments, an antibody of the disclosure is a recombinant antibody, particularly a humanized monoclonal antibody or a fully human recombinant antibody.

The adjective “specifically reactive with” as used in reference to an antibody is intended to mean, as is generally understood in the art, that the antibody is sufficiently selective between the antigen of interest (e.g. an ALK1 polypeptide or an ALK1 ligand) and other antigens that are not of interest that the antibody is useful for, at minimum, detecting the presence of the antigen of interest in a particular type of biological sample. In certain methods employing the antibody, a higher degree of specificity in binding may be desirable. For example, an antibody for use in detecting a low abundance protein of interest in the presence of one or more very high abundance protein that are not of interest may perform better if it has a higher degree of selectivity between the antigen of interest and other cross-reactants. Monoclonal antibodies generally have a greater tendency (as compared to polyclonal antibodies) to discriminate effectively between the desired antigens and cross-reacting polypeptides. In addition, an antibody that is effective at selectively identifying an antigen of interest in one type of biological sample (e.g. a stool sample) may not be as effective for selectively identifying the same antigen in a different type of biological sample (e.g. a blood sample). Likewise, an antibody that is effective at identifying an antigen of interest in a purified protein preparation that is devoid of other biological contaminants may not be as effective at identifying an antigen of interest in a crude biological sample, such as a blood or urine sample. Accordingly, in preferred embodiments, the application provides antibodies that have demonstrated specificity for an antigen of interest in a sample type that is likely to be the sample type of choice for use of the antibody.

One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less. Given the apparently low binding affinity of TGFβ for ALK1, it is expected that many anti-ALK1 antibodies will inhibit TGFβ binding. However, the BMP9 and BMP10 ligands bind ALK1 with a K_{D} of approximately 1×10⁻¹⁰ M. Thus, antibodies of appropriate affinity may be selected to interfere with the signaling activities of these ligands.

In addition, the techniques used to screen antibodies in order to identify a desirable antibody may influence the properties of the antibody obtained. For example, an antibody to be used for certain therapeutic purposes will preferably be able to target a particular cell type. Accordingly, to obtain antibodies of this type, it may be desirable to screen for antibodies that bind to cells that express the antigen of interest (e.g. by fluorescence activated cell sorting). Likewise, if an antibody is to be used for binding an antigen in solution, it may be desirable to test solution binding. A variety of different techniques are available for testing antibody:antigen interactions to identify particularly desirable antibodies. Such techniques include ELISAs, surface plasmon resonance binding assays (e.g. the Biacore binding assay, Bia-core AB, Uppsala, Sweden), sandwich assays (e.g. the paramagnetic bead system of IGEN International, Inc., Gaithersburg, Md.), western blots, immunoprecipitation assays and immunohistochemistry.

Another aspect of the disclosure pertains to a ALK1 ligand antagonistic antibodies. These include antibodies that disrupt the binding of an ALK1 ligand, e.g., of BMP9 or BMP10 to one or more of its receptors, e.g., to ALK1 or endoglin. Accordingly, some ALK1 ligand antagonistic antibodies may also be ALK1 antagonistic antibodies. In some embodiments, the ALK1 ligand-antagonistic antibody is an antibody reactive with an extracellular portion of an ALK1 ligand receptor, e.g., a receptor that binds to an ALK1 ligand. In some embodiments, the ALK1 ligand-antagonistic antibody is an antibody reactive with an extracellular portion of an ALK1 polypeptide, preferably an antibody that specifically binds to an ALK1 polypeptide ECD as described herein. In some embodiments, the ALK1 ligand-antagonistic antibody is an antibody reactive with an extracellular portion of an endoglin polypeptide, preferably an antibody that specifically binds to an endoglin polypeptide ECD as described herein. In a preferred embodiment, such antibody interferes with receptor binding to the ligand such as BMP9 and/or BMP10. In other embodiments, the ALK1 ligand-antagonistic antibody is an antibody reactive with an ALK1 ligand, e.g., with BMP9 and/or BMP10, and interferes with the binding of the ligand to one or more of the ligands receptors. In a preferred embodiment, such antibody interferes with the binding of all receptors that to the ligand, e.g. with the binding of ALK1 and endoglin to BMP9 and/or BMP10. It will be understood that an ALK1 ligand-antagonistic antibody, to be functional in vivo, should bind to the mature, processed form of the relevant protein. e.g., the ALK1 protein, the endoglin protein, or the respective ligand, e.g., BMP9 or BMP10. Preferred antibodies are those that exhibit an anti-angiogenic activity in a bioassay, such as a CAM assay or corneal micropocket assay. Some ALK1 ligand-antagonistic antibodies are described herein and additional ALK1 ligand-antagonistic antibodies are known to those of skill in the art, and include, without limitation, the endoglin antibodies described in U.S. Pat. No. 8,221,753, the entire contents of which are incorporated herein by reference.

E. Alterations in Antibodies and Fc Fusion Proteins

The application further provides antibodies and, ALK1-Fc fusion proteins with engineered or variant Fc regions. Such antibodies and Fc fusion proteins may be useful, for example, in modulating effector functions, such as, antigen-dependent cytotoxicity (ADCC) and complement-dependent cytotoxicity (CDC). Additionally, the modifications may improve the stability of the antibodies and Fc fusion proteins. Amino acid sequence variants of the antibodies and Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the antibodies and Fc fusion proteins, such as changing the number or position of glycosylation sites.

Antibodies and Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26). Thus in certain embodiments, antibodies and Fc fusion proteins of the disclosure with mutations within the constant region including the Ala-Ala mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation would describe a mutation(s) from phenylalanine to alanine at position 234 and/or a mutation from leucine to alanine at position 235. In another embodiment, the antibody or Fc fusion protein comprises an IgG1 framework, wherein the Ala-Ala mutation would describe a mutation(s) from leucine to alanine at position 234 and/or a mutation from leucine to alanine at position 235. The antibody or Fc fusion protein may alternatively or additionally carry other mutations, including the point mutation K322A in the CH2 domain (Hezareh et al. 2001 J Virol. 75: 12161-8).

In particular embodiments, the antibody or Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. No. 5,648,260; U.S. Pat. No. 5,624,821; and WO94/29351.

F. Nucleic Acid ALK1 Antagonists

In certain aspects, the disclosure provides ALK1 antagonists that comprise isolated and/or recombinant nucleic acids encoding any of the ALK1 antagonists (e.g., ALK1 ECD polypeptides, ALK1-Fc fusion proteins), including fragments, functional variants and fusion proteins disclosed herein. For example, SEQ ID NO: 2 encodes the naturally occurring human ALK1 precursor polypeptide, while SEQ ID NO: 4 encodes the precursor of an ALK1 extracellular domain fused to an IgG1 Fc domain. The subject nucleic acids may be single-stranded or double stranded. Such nucleic acids may be DNA or RNA molecules. These nucleic acids may be used, for example, in methods for making ALK1 polypeptides or as direct therapeutic ALK1 antagonists (e.g., in an antisense, RNAi, or gene therapy approaches).

In certain aspects, the subject nucleic acids encoding ALK1 antagonists are further understood to include nucleic acids that are variants of SEQ ID NO: 2 or 4. Variant nucleotide sequences include sequences that differ by one or more nucleotide substitutions, additions or deletions, such as allelic variants.

In certain embodiments, the disclosure provides isolated or recombinant nucleic acid sequences that are at least 80%, 85%, 90%, 95%, 96%, 97%, 98%, 99% or 100% identical to SEQ ID NO: 2 or 4. One of ordinary skill in the art will appreciate that nucleic acid sequences complementary to SEQ ID NO: 2 or 4, and variants of SEQ ID NO: 2 or 4 are also within the scope of this disclosure. In further embodiments, the nucleic acid sequences of the disclosure can be isolated, recombinant, and/or fused with a heterologous nucleotide sequence, or in a DNA library.

In other embodiments, nucleic acids of the disclosure also include nucleotide sequences that hybridize under highly stringent conditions to the nucleotide sequence designated in SEQ ID NO: 2 or 4, complement sequence of SEQ ID NO: 2 or 4, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 2 or 4 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects disclosed herein, the subject nucleic acid is provided in an expression vector comprising a nucleotide sequence encoding an ALK1 antagonists and operably linked to at least one regulatory sequence. Regulatory sequences are art-recognized and are selected to direct expression of the ALK1 antagonists. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding an ALK1 antagonist. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid included in the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ALK1 antagonist include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Ban virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In a preferred embodiment, a vector will be designed for production of the subject ALK1 antagonists in CHO cells, such as a Pcmv-Script vector (Stratagene, La Jolla, Calif.), pcDNA4 vectors (Invitrogen, Carlsbad, Calif.) and pCI-neo vectors (Promega, Madison, Wis.). As will be apparent, the subject gene constructs can be used to cause expression of the subject ALK1 polypeptides in cells propagated in culture, e.g., to produce proteins, including fusion proteins or variant proteins, for purification.

This disclosure also pertains to a host cell transfected with a recombinant gene including a coding sequence (e.g., SEQ ID NO: 2 or 4) for one or more of the subject ALK1 antagonists. The host cell may be any prokaryotic or eukaryotic cell. For example, an ALK1 polypeptide disclosed herein may be expressed in bacterial cells such as E. coli, insect cells (e.g., using a baculovirus expression system), yeast, or mammalian cells. Other suitable host cells are known to those skilled in the art.

Examples of categories of nucleic acid compounds that are antagonists of ALK1 include antisense nucleic acids, RNAi constructs and catalytic nucleic acid constructs. A nucleic acid compound may be single or double stranded. A double stranded compound may also include regions of overhang or non-complementarity, where one or the other of the strands is single stranded. A single stranded compound may include regions of self-complementarity, meaning that the compound forms a so-called “hairpin” or “stem-loop” structure, with a region of double helical structure. A nucleic acid compound may comprise a nucleotide sequence that is complementary to a region consisting of no more than 1000, no more than 500, no more than 250, no more than 100 or no more than 50, 35, 30, 25, 22, 20 or 18 nucleotides of the full-length ALK1 nucleic acid sequence or ligand nucleic acid sequence. The region of complementarity will preferably be at least 8 nucleotides, and optionally at least 10 or at least 15 nucleotides, and optionally between 15 and 25 nucleotides. A region of complementarity may fall within an intron, a coding sequence or a noncoding sequence of the target transcript, such as the coding sequence portion. Generally, a nucleic acid compound will have a length of about 8 to about 500 nucleotides or base pairs in length, and optionally the length will be about 14 to about 50 nucleotides.

A nucleic acid may be a DNA (particularly for use as an antisense), RNA or RNA:DNA hybrid. Any one strand may include a mixture of DNA and RNA, as well as modified forms that cannot readily be classified as either DNA or RNA. Likewise, a double stranded compound may be DNA:DNA, DNA:RNA or RNA:RNA, and any one strand may also include a mixture of DNA and RNA, as well as modified forms that cannot readily be classified as either DNA or RNA. A nucleic acid compound may include any of a variety of modifications, including one or modifications to the backbone (the sugar-phosphate portion in a natural nucleic acid, including internucleotide linkages) or the base portion (the purine or pyrimidine portion of a natural nucleic acid). An antisense nucleic acid compound will preferably have a length of about 15 to about 30 nucleotides and will often contain one or more modifications to improve characteristics such as stability in the serum, in a cell or in a place where the compound is likely to be delivered, such as the stomach in the case of orally delivered compounds and the lung for inhaled compounds. In the case of an RNAi construct, the strand complementary to the target transcript will generally be RNA or modifications thereof. The other strand may be RNA, DNA or any other variation. The duplex portion of double stranded or single stranded “hairpin” RNAi construct will preferably have a length of 18 to 40 nucleotides in length and optionally about 21 to 23 nucleotides in length, so long as it serves as a Dicer substrate. Catalytic or enzymatic nucleic acids may be ribozymes or DNA enzymes and may also contain modified forms. Nucleic acid compounds may inhibit expression of the target by about 50%, 75%, 90% or more when contacted with cells under physiological conditions and at a concentration where a nonsense or sense control has little or no effect. Preferred concentrations for testing the effect of nucleic acid compounds are 1, 5 and 10 micromolar. Nucleic acid compounds may also be tested for effects on, for example, angiogenesis.

G. ALK1 Ligand Pro Peptides

Some aspects of this disclosure provide pro-peptides of ALK1 ligands as ALK1 antagonists useful in the methods and kits provided herein. Accordingly, some aspects provide methods using ALK1 ligand pro-peptides, e.g., BMP9 and/or BMP10 pro-peptides as ALK1 antagonists, e.g., in the treatment of cancer.

The ALK1 ligands BMP9 and BMP10 are typically generated as larger precursor proteins of the general structure N-[cleavage site]-[pro-peptide]-[cleavage site]-[active BMP polypeptide]-C. It has been shown that BMP pro-proteins interfere with the binding of the mature BMP polypeptide to it respective receptor(s), e.g., the binding of BMP9 and BMP10 to ALK1.

For example, the human BMP10 gene encodes a precursor protein of the sequence provided below (SEQ ID NO: 11):

>gi|13123977|sp|095393.1|BMP10_HUMAN RecName: Full = Bone
morphogenetic protein 10; Short = BMP-10; Flags: Precursor
MGSLVLTLCALFCLAAYLVSGSPIMNLEQSPLEEDMSLFGDVFSEQDGVDFNTLLQSMKDEFLKTLNLS
DIPTQDSAKVDPPEYMLELYNKFATDRTSMPSANIIRSFKNEDLFSQPVSFNGLRKYPLLFNVSIPHHE
EVIMAELRLYTLVQRDRMIYDGVDRKITIFEVLESKGDNEGERNMLVLVSGEIYGTNSEWETFDVTDAI
RRWQKSGSSTHQLEVHIESKHDEAEDASSGRLEIDTSAQNKHNPLLIVFSDDQSSDKERKEELNEMISH
EQLPELDNLGLDSFSSGPGEEALLQMRSNIIYDSTARIRRNAKGNYCKRTPLYIDFKEIGWDSWIIAPP
GYEAYECRGVCNYPLAEHLTPTKHAIIQALVHLKNSQKASKACCVPTKLEPISILYLDKGVVTYKFKYE
GMAVSECGCR

In the sequence above, the BMP10 pro-peptide is underlined. The N-terminal 21 amino acids constitute the signal peptide and the C-terminal 108 amino acids constitute the mature, active BMP10 polypeptide that binds to ALK1 once cleaved from the N-terminal sequences.

Similarly, the human BMP9 gene encodes a precursor protein of the sequence provided below (SEQ ID NO: 10):

>gi|13124266|sp|Q9UK05.1|GDF2_HUMAN RecName:
Full = Growth/differentiation factor 2; Short = GDF-2; AltName:
Full = Bone morphogenetic protein 9; Short = BMP-9; Flags: Precursor
MCPGALWVALPLLSLLAGSLQGKPLQSWGRGSAGGNAHSPLGVPGGGLPEHTFNLKMFLENVKVDFLRS
LNLSGVPSQDKTRVEPPQYMIDLYNRYTSDKSTTPASNIVRSFSMEDAISITATEDFPFQKHILLFNIS
IPRHEQITRAELRLYVSCQNHVDPSHDLKGSVVIYDVLDGTDAWDSATETKTFLVSQDIQDEGWETLEV
SSAVKRWVRSDSTKSKNKLEVTVESHRKGCDTLDISVPPGSRNLPFFVVFSNDHSSGTKETRLELREMI
SHEQESVLKKLSKDGSTEAGESSHEEDTDGHVAAGSTLARRKRSAGAGSHCQKTSLRVNFEDIGWDSWI
IAPKEYEAYECKGGCFFPLADDVTPTKHAIVQTLVHLKFPTKVGKACCVPTKLSPISVLYKDDMGVPTL
KYHYEGMSVAECGCR

In the sequence above, the BMP9 pro-peptide is underlined. The N-terminal 22 amino acids constitute the signal peptide and the C-terminal 103 amino acids constitute the mature, active BMP9 polypeptide that binds to ALK1 once cleaved from the N-terminal sequences.

Additional ALK1 ligand pro-peptides are known to those of skill in the art, including, for example, those disclosed in PCT Application Publication WO/2008/151078 and in U.S. Pat. No. 7,741,284, the entire contents of which are incorporated herein by reference. It will be understood that the specific ALK1 ligand pro-proteins are exemplary and that the invention is not limited in this respect.

Some aspects of this disclosure provide that ALK1 ligand pro-peptides function as ALK1 antagonists. Without wishing to be bound by any specific theory, it is believed that the pro-proteins inhibit or disrupt binding of the mature ALK1 ligand to the receptor, for example, by competitive association with the ligand or the receptor. Accordingly, in some embodiments, an ALK1 ligand pro-protein, e.g., a BMP10 pro-protein and/or the BMP9 pro-protein, is used as an ALK1 antagonist. For example, in some embodiments, an ALK1 ligand pro-protein, e.g., a BMP10 pro-peptide or a BMP9 pro-peptide, is administered to a subject, e.g., a subject having a cancer identified to be responsive to treatment with an ALK1 antagonist, in an amount effective to treat the cancer.

ALK1 ligand pro-peptides include fragments, functional variants, and modified forms (e.g. peptidomimetic forms) of naturally occurring pro-peptides. In some embodiments, a pro-peptide does not include a full-length mature ALK1 ligand polypeptide, but may include a portion of the mature ligand domain.

### 5. Formulations and Effective Doses

The ALK1 antagonists described herein may be formulated into pharmaceutical compositions. Pharmaceutical compositions for use in accordance with the present disclosure may be formulated in conventional manner using one or more physiologically acceptable carriers or excipients. Such formulations will generally be substantially pyrogen free, in compliance with most regulatory requirements.

In certain embodiments, the therapeutic method of the disclosure includes administering the composition systemically, or locally as an implant or device. Therapeutically useful agents other than the ALK1 antagonists which may also optionally be included in the composition as described above, may be administered simultaneously or sequentially with the subject compounds (e.g., ALK1 ECD polypeptides or any of the antibodies disclosed herein) in the methods disclosed herein.

Typically, ALK1 antagonists disclosed herein will be administered parentally, and particularly intravenously or subcutaneously. Pharmaceutical compositions suitable for parenteral administration may comprise one or more ALK1 antagonists in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In one embodiment, the ALK1 antagonists disclosed herein are administered in an ophthalmic pharmaceutical formulation. In some embodiments, the ophthalmic pharmaceutical formulation is a sterile aqueous solution, preferable of suitable concentration for injection, or a salve or ointment. Such salves or ointments typically comprise one or more ALK1 antagonists disclosed herein dissolved or suspended in a sterile pharmaceutically acceptable salve or ointment base, such as a mineral oil-white petrolatum base. In salve or ointment compositions, anhydrous lanolin may also be included in the formulation. Thimerosal or chlorobutanol are also preferably added to such ointment compositions as antimicrobial agents. In one embodiment, the sterile aqueous solution is as described in U.S. Pat. No. 6,071,958.

The disclosure provides formulations that may be varied to include acids and bases to adjust the pH; and buffering agents to keep the pH within a narrow range. Additional medicaments may be added to the formulation. These include, but are not limited to, pegaptanib, heparinase, ranibizumab, or glucocorticoids. The ophthalmic pharmaceutical formulation according to the disclosure is prepared by aseptic manipulation, or sterilization is performed at a suitable stage of preparation.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### EXAMPLES

### Example 1

### Expression of ALK1-Fc Fusion Proteins

A soluble ALK1 fusion protein was constructed that has the extracellular domain of human ALK1 fused to a human Fc or mouse ALK1 fused to a murine Fc domain with a minimal linker in between. The constructs are referred to as hALK1-Fc and mALK1-Fc, respectively.

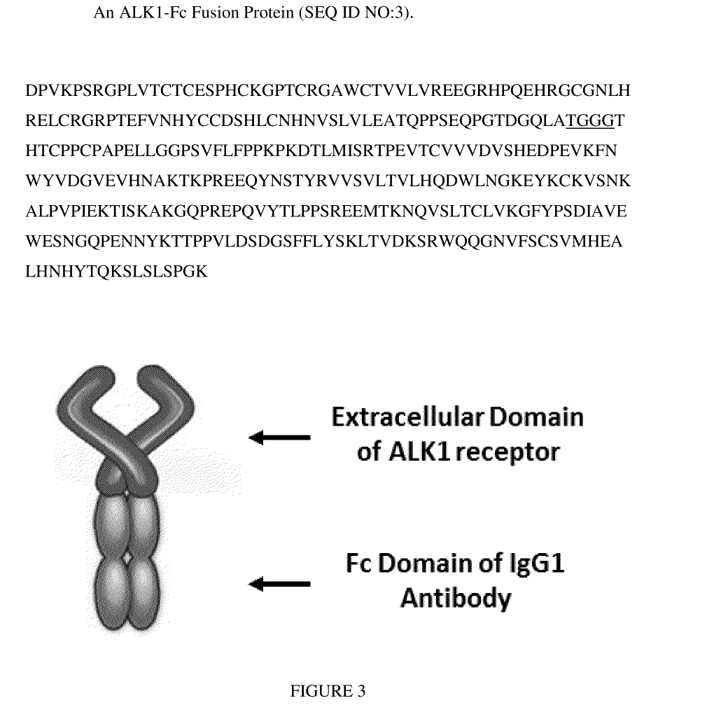

hALK1-Fc is shown as purified from CHO cell lines in FIG. 3 (SEQ ID NO: 3). Notably, while the conventional C-terminus of the extracellular domain of human ALK1 protein is amino acid 118 of SEQ ID NO:1, we have determined that it is desirable to avoid having a domain that ends at a glutamine residue. Accordingly, the portion of SEQ ID NO:3 that derives from human ALK1 incorporates two residues c-terminal to Q118, a leucine and an alanine. The disclosure therefore provides ALK1 ECD polypeptides (including Fc fusion proteins) having a c-terminus of the ALK1 derived sequence that is anywhere from 1 to 5 amino acids upstream (113-117 relative to SEQ ID NO:1) or downstream (119-123) of Q118.

The hALK1-Fc and mALK1-Fc proteins were expressed in CHO cell lines. Three different leader sequences were considered:

(i) Honey bee mellitin (HBML):
(SEQ ID NO: 7)
MKFLVNVALVFMVVYISYIYA
(ii) Tissue Plasminogen Activator (TPA):
(SEQ ID NO: 8)
MDAMKRGLCCVLLLCGAVFVSP
(iii) Native:
(SEQ ID NO: 9)
MTLGSPRKGLLMLLMALVTQG.

Purification can be achieved by a series of column chromatography steps, including, for example, three or more of the following, in any order: protein A chromatography, Q sepharose chromatography, phenylsepharose chromatography, size exclusion chromatography, and cation exchange chromatography. The purification can be completed with viral filtration and buffer exchange. The hALK1-Fc protein was purified to a purity of >98% as determined by size exclusion chromatography and >95% as determined by SDS PAGE.

In the course of protein production and purification, we observed that hALK1-Fc tends to be expressed in a mixture of dimers and higher order aggregates which, while appearing pure under denaturing, reducing conditions (e.g., reducing SDS-PAGE), are problematic for administration to a patient. The aggregates may be immunogenic or poorly bioavailable, and because of their heterogeneity, these aggregates make it difficult to characterize the pharmaceutical preparation at a level that is desirable for drug development. Thus, various approaches were tested to reduce the amount of aggregate in final preparations.

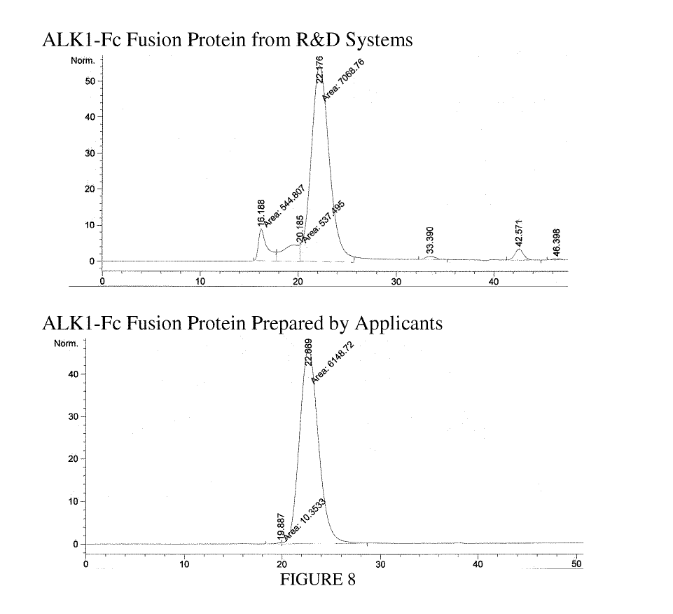

In one approach, a number of different cell culture media were tested. IS CHO-CD (Cat. No. 91119, Irvine Scientific, Santa Ana, Calif.) showed a remarkable reduction in the production of aggregated products, while maintaining high level production of the hALK1-Fc. Additionally, elution of the material from a hydrophobic interaction column (e.g., phenylsepharose) at a pH of 8.0 resulted in further resolution of the aggregated product. The resulting material is comprised of greater than 99% dimers. A comparison to an ALK1-Fc fusion protein sold by R&D Systems (cat. no. 370-AL, Minneapolis, Minn.) shows that this protein, produced in NSO cells, is 84% dimers, with the remaining protein appearing as high molecular weight species by size exclusion chromatography. A comparison of the sizing column profile for the preparations is shown in FIG. 8. Having identified aggregate formation as a significant problem in ALK1-Fc production, it is expected that other approaches may be developed, including approaches that involve additional purification steps (although such approaches may result in lower yield of purified protein).

### Example 2

### Identification of ALK1-Fc Ligands

ALK1 is a type 1 receptors for members of the TGFβ family. A variety of members of the TGFβ family were tested for binding to a human ALK1-Fc fusion protein, using a Biacore™ system. TGFβ itself, GDF8, GDF11, BMP2 and BMP4 all failed to show substantial binding to the hALK1-Fc protein. BMP2 and BMP4 showed limited binding. GDF5, GDF7 and BMP9 showed binding with K_{D} values of approximately 5×10⁻⁸ M, 5×10⁻⁸ M and 1×10⁻¹⁰ M, respectively. Based on the similarity of GDF5 and GDF7 to GDF6, it is expected that GDF6 will bind with similar affinity. BMP10 is closely related to BMP9 and is also expected to bind with similar affinity.

### Example 3

### Characterization of ALK1-Fc and Anti-ALK1 Antibody Effects on Endothelial Cells

Using a luciferase reporter construct under the control of four sequential consensus SBE sites (SBE4-luc), which are responsive to Smad1/5/8-mediated signaling, we measured BMP-9 mediated activity in the presence and absence of hALK1-Fc drug or neutralizing ALK1 specific monoclonal antibody in HMVEC cells. HMVEC cells were stimulated with rhBMP-9 (50 ng/ml), which induced Smad1/5/8-mediated transcriptional activation, evidenced here by the increase in SBE4-luc modulated transcriptional upregulation. When added, the hALK1-Fc compound (10 μg/ml) or antibody (10 μg/ml) diminished this transcriptional response, each by nearly 60%, indicating that the presence of ALK1-Fc significantly reduces BMP9 signaling, and moreover, that the BMP9 signaling is related to ALK1 activity.

Activation of SMAD phosphorylation is commonly used to assay activation of upstream activin receptors. ALK1 is known to modulate phosphorylation of SMAD proteins 1, 5 and 8 upon activation by its ligand. Here, we added rhBMP-9 (50 ng/ml) to initiate SMAD phosphorylation in HUVEC cells, a human endothelial cell line which innately expresses ALK1 receptor, over a time course of 30 minutes. Phosphorylation of SMAD 1/5/8 was seen 5 minutes after treatment of cells with ligand and phosphorylation was maintained for the entirety of the 30 minute period. In the presence of relatively low concentrations of hALK1-Fc (250 ng/ml), SMAD 1/5/8 phosphorylation was reduced, confirming that this agent inhibits Smad1/5/8 activation in endothelial cells.

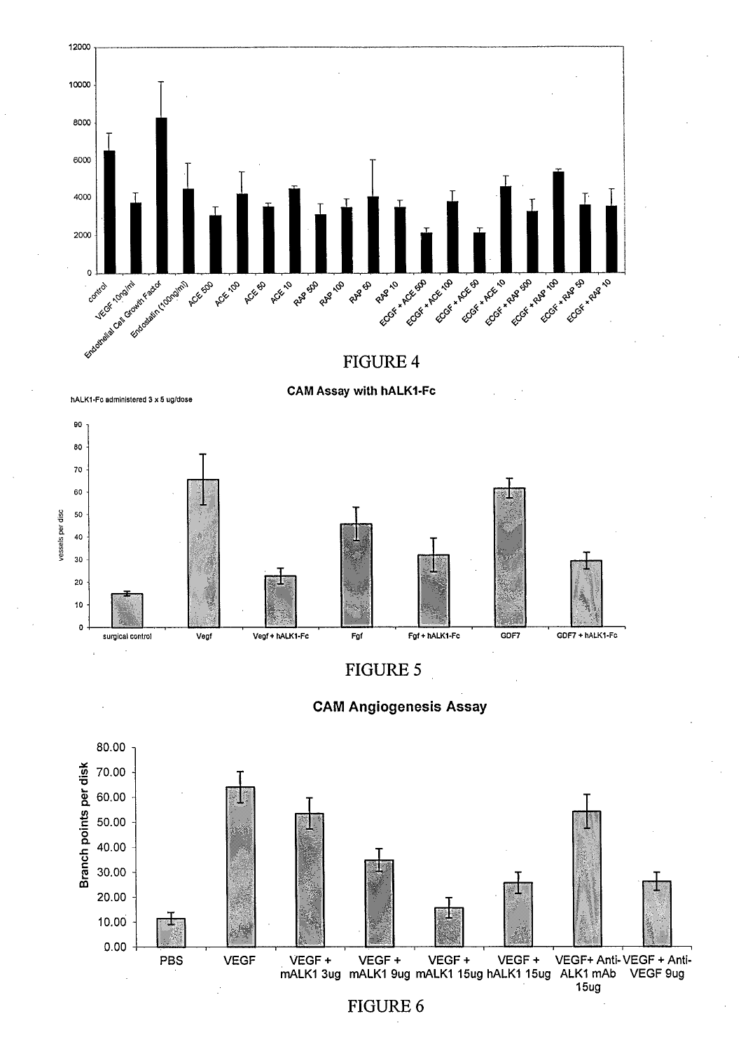

In order to evaluate the angiogenic effect of ALK1-Fc in an in vitro system, we assayed the effectiveness of the compound in reducing tube formation of endothelial cells on a Matrigel substrate. This technique is commonly used to assess neovascularization, giving both rapid and highly reproducible results. Endothelial Cell Growth Supplement (ECGS) is used to induce the formation of microvessels from endothelial cells on Matrigel, and the efficacy of anti-angiogenic compounds are then gauged as a reduction of cord formation in the presence of both the drug and ECGS over an 18 hour time course. As expected, addition of ECGS (200 ng/ml) induced significant cord formation, as compared to the negative control (no treatment added), which indicates basal levels of endothelial cell cord formation produced on Matrigel substrate (FIG. 4). Upon addition of either hALK1-Fc (100 ng/ml) or mALK1-Fc (100 ng/ml), cord formation was visibly reduced. Final quantification of vessel length in all samples revealed that every concentration of hALK1-fc or mALK1-Fc reduced neovascularization to basal levels. Additionally, hALK1-Fc and mALK1-Fc in the presence of the strongly pro-angiogenic factor ECGS maintained strong inhibition of neovascularization demonstrating even more potent anti-angiogenic activity than the negative control endostatin (100 ng/ml).

### Example 4

### CAM Assays

VEGF and FGF are well-known to stimulate angiogenesis. A CAM (chick chorioallantoic membrane) assay system was used to assess the angiogenic effects of GDF7. GDF7 stimulates angiogenesis with a potency that is similar to that of VEGF. Similar results were observed with GDF5 and GDF6.

ALK1-Fc fusions were tested for anti-angiogenic activity in the CAM assay. These fusion proteins showed a potent anti-angiogenic effect on angiogenesis stimulated by VEGF, FGF and GDF7. See FIG. 5. BMP9 and PDGF showed a relatively poor capability to induce angiogenesis in this assay, but such angiogenic effect of these factors was nonetheless inhibited by ALK1.

ALK1-Fc proteins and a commercially available, anti-angiogenic anti-VEGF monoclonal antibody were compared in the CAM assay. The ALK1-Fc proteins had similar potency as compared to anti-VEGF. The anti-VEGF antibody bevacizumab is currently used in the treatment of cancer and macular degeneration in humans. See FIG. 6.

Interestingly, an anti-ALK1 antibody (R&D Systems) failed to significantly inhibit angiogenesis in this assay system. We expect that this may reflect the difference in the ALK1 sequence in different species.

### Example 5

### Mouse Corneal Micropocket Assay

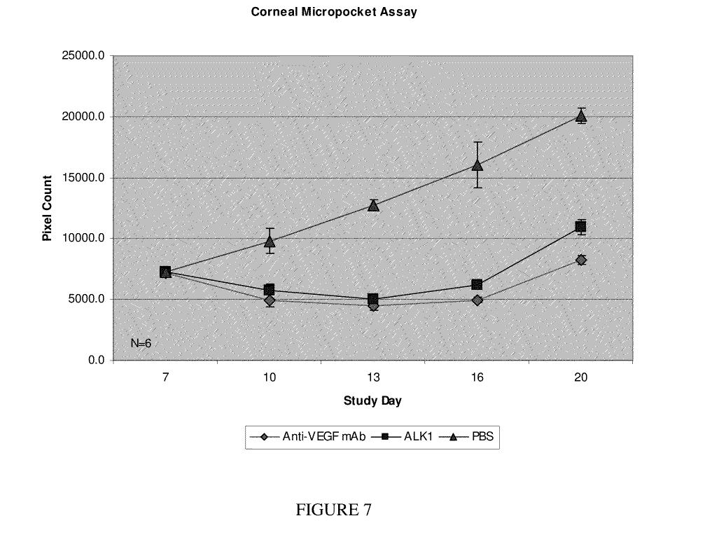

The mouse corneal micropocket assay was used to assess the effects of ALK1-Fc on angiogenesis in the mouse eye. hALK1-Fc, administered intraperitoneally, significantly inhibited ocular angiogenesis. As shown in FIG. 7, hALK1-Fc inhibited ocular angiogenesis to the same degree as anti-VEGF. hALK1-Fc and anti-VEGF were used at identical weight/weight dosages. Similar data were obtained when a Matrigel plug impregnated with VEGF was implanted in a non-ocular location.

These data demonstrate that high affinity ligands for ALK1 promote angiogenesis and that an ALK1-Fc fusion protein has potent anti-angiogenic activity. The ligands for ALK1 fall into two categories, with the GDF5, 6, 7 grouping having an intermediate affinity for ALK1 and the BMP9, 10 grouping having a high affinity for ALK1.

GDF5, 6 and 7 are primarily localized to bone and joints, while BMP9 is circulated in the blood. Thus, there appears to be a pro-angiogenic system of the bones and joints that includes ALK1, GDF5, 6 and 7 and a systemic angiogenic system that includes ALK1 and BMP9 (and possibly BMP10).

### Example 6

### ALK1-Fc Reduces Tumor Angiogenesis in a CAM Assay

Tumors, as with any tissue, have a basic nutrient and oxygen requirement. Although small tumors are capable of acquiring adequate amounts via diffusion from neighboring blood vessels, as the tumor increases in size, it must secure nutrients by recruiting and maintaining existing capillaries. In order to test the capacity of ALK1-Fc proteins to limit tumor growth through vessel inhibition, we tested varying concentrations of mALK1-Fc in a melanoma explant CAM assay. As with CAM assays described above, small windows were made in the surface of each egg through which 5×10⁵ B16 melanoma cells were implanted. Eggs were then treated daily with 0.02 mg/ml mALK1-Fc, 0.2 mg/ml mALK1-Fc, or left untreated for a period of a week. At the end of the experiment, tumors were carefully removed, weighed and digital images were captured. Tumors originating from CAMs treated with mALK1-Fc showed a significant decrease in size as compared to untreated CAM tumors. Quantification of tumor weight demonstrated that weight of tumors treated daily with either 0.02 mg/ml or 0.2 mg/ml mALK1-Fc showed a reduction of 65% and 85% compared to the untreated CAMs. In conclusion, neovascularization and tumor growth was significantly suppressed upon addition of ALK1-Fc in a dose-responsive manner, indicating that ALK1-Fc is a powerful anti-angiogenic agent.

### Example 7

### Lung Cancer Experimental Model

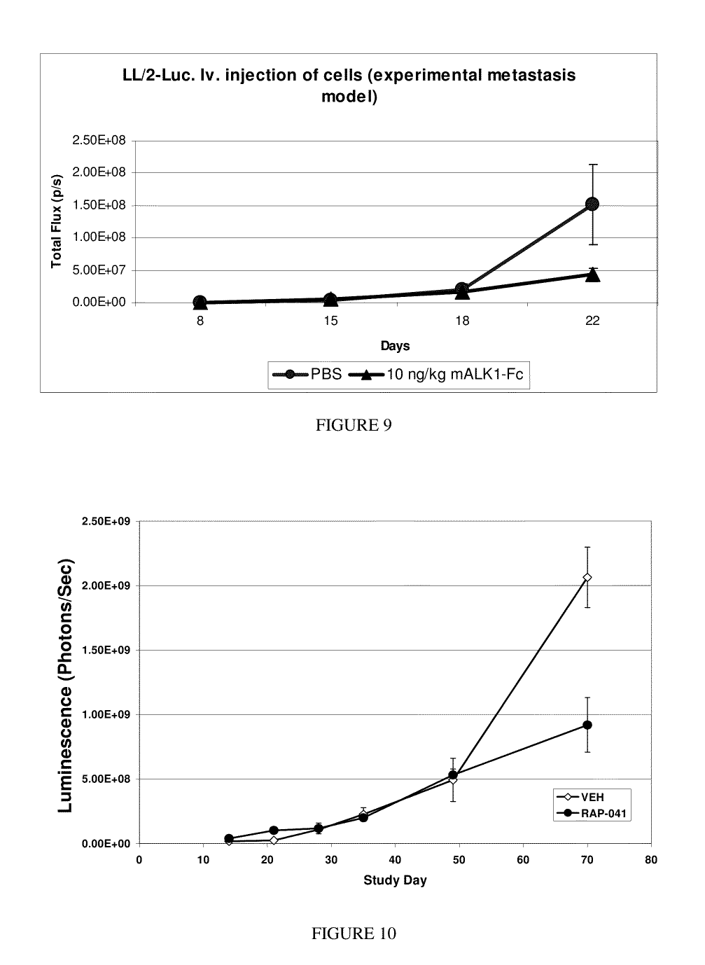

To further confirm the effects of ALK1-Fc on tumor progression, a mouse model of lung cancer was tested. Fluorescently labeled murine Lewis lung cancer cells (LL/2-luc) were administered to albino Black 6 mice through the tail vein. On the same day, the mice began treatment with either PBS control (n=7) or 10 mg/kg mALK1-Fc (n=7) administered intraperitoneally. In-life fluorescent imaging showed substantial development of tumors localized to the lungs in the control mice, to the point that the mice became moribund and had to be sacrificed by day 22 post-implantation. By contrast, the ALK1-Fc treated mice showed a substantially delayed growth of lung tumors and exhibited 100% survival as of day 22. See FIG. 9.

These data demonstrate that ALK1-Fc has substantial effect on tumor growth in a mouse model of lung cancer and provides a survival benefit.

### Example 8

### Effects of ALK1-Fc Fusion Protein on Breast Cancer Tumor Models

mALK1-Fc was effective in delaying the growth of breast cancer tumor cell lines derived from both estrogen receptor positive (ER+) and estrogen receptor negative tumor cells (ER−).

The MDA-MB-231 breast cancer cell line (derived from ER− cells) was stably transfected with the luciferase gene to allow for the in vivo detection of tumor growth and potential metastasis. In this study, 1×10⁶ MDA-MB-231-Luc cells were implanted orthotopically in the mammary fat pad of athymic nude mice (Harlan). Tumor progression was followed by bioluminescent detection using an IVIS Spectrum imaging system (Caliper Life Sciences). An increase in the luminescence (number of photons detected) corresponds to an increase in tumor burden.

Thirty female nude mice were injected with 1×10⁶ tumor cells into the mammary fat pad. Three days after tumor implantation the mice were treated with either vehicle control or mALK1-Fc (30 mg/kg) twice per week by subcutaneous (SC) injection. Treatment was continued and tumor progression was monitored by bioluminescent imaging for 10 weeks. mALK1-Fc treatment at 30 mg/kg slowed tumor progression as determined by bioluminescent detection when compared to vehicle treated controls (FIG. 10). Treatment with mALK1-Fc delayed, but did not reverse tumor growth in this model. This may be expected of an antiangiogenic compound in that tumors may be able to survive to a certain size before requiring new blood vessel formation to support continued growth. In a similar experiment, hALK1-Fc produced similar, if slightly lesser, effects at dose levels as low as 3 mg/kg.

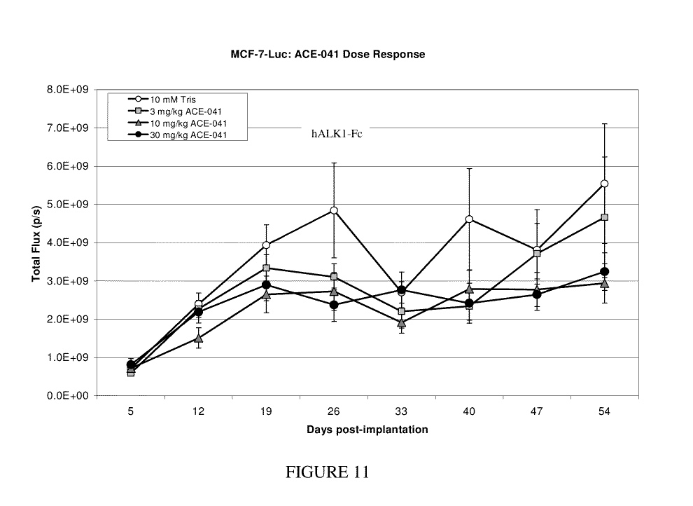

The estrogen-receptor-positive (ER+), luciferase expressing cell line, MCF-7, was also tested in an orthotopic implantation model. In this model, female nude mice are implanted subcutaneously with a 60 day slow release pellet of 17β-estradiol. Two days following pellet implantation, 5×10⁶ MCF-7 tumor cells were implanted into the mammary fat pad. Mice were treated twice per week with hALK1-Fc at 3, 10 and 30 mg/kg, or vehicle control, by the IP route. Tumor progression was followed by bioluminescent imaging on a weekly basis with an IVIS-Spectrum imager (Caliper Life Sciences). In vehicle treated mice tumors progressed rapidly until study day 26 (FIG. 11). After day 26 there were fluctuations in tumor luminescence until the conclusion of the study at day 60 (when the estradiol pellets were depleted). These fluctuations are due to a common feature of this model in that the rapid tumor growth can exceed the angiogenic response of the host animals leading to tumor necrosis and a concomitant drop-off in luminescent signal. The remaining cells continue to grow leading to an increased signal. Mice treated with 10 or 30 mg/kg of hALK1-Fc were able to maintain tumor size at a constant level during the study, compared to vehicle-treated controls, indicating a potent effect of this molecule on tumor growth.

### Example 9

### Expression of BMP9 and BMP10 in Cancer

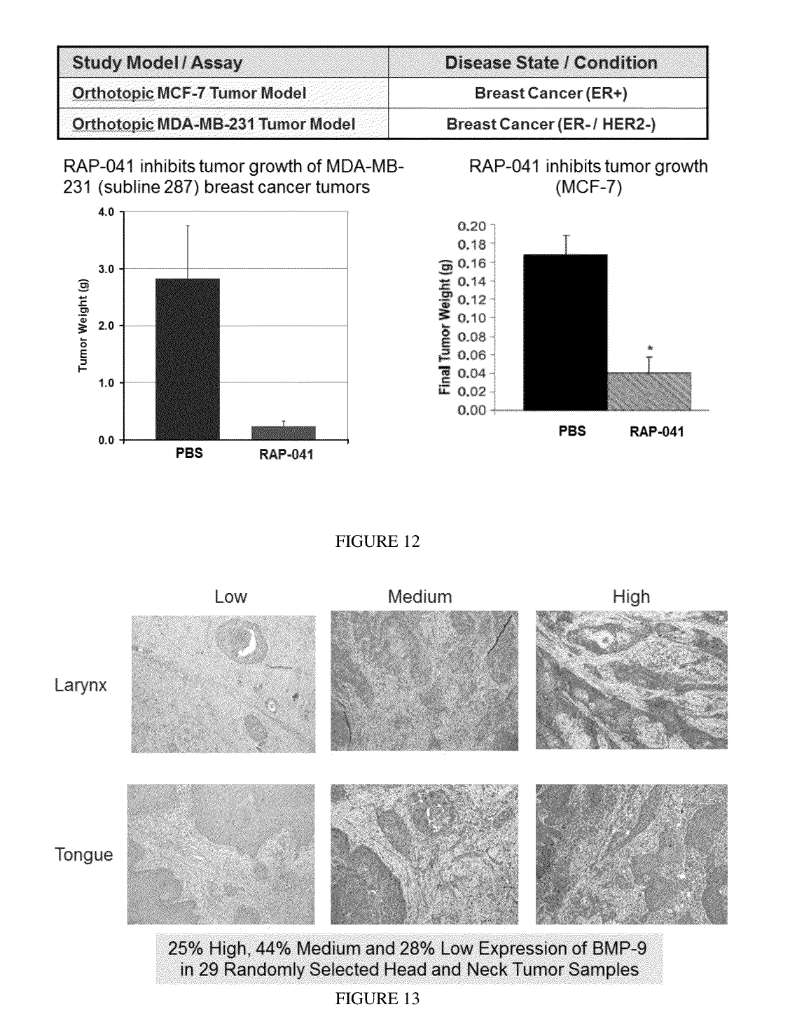

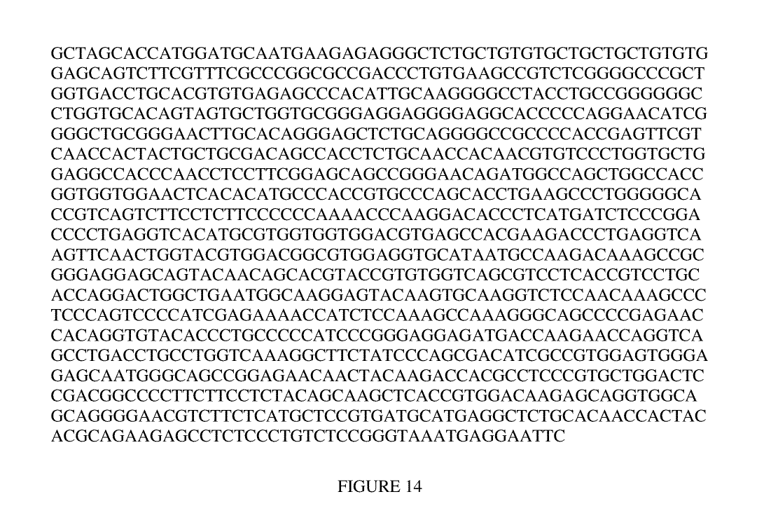

Efficacy of ALK1-Fc (SEQ ID NO: 3, RAP-041) was tested in orthotopic animal models. As shown in FIG. 12, ALK1-Fc was observed to significantly reduce tumor growth in orthotopic models MCF-7 (breast cancer ER+) and MDA-MB-231 (breast cancer ER−/HER2−).

Efficacy of ALK1-Fc (SEQ ID NO: 3) was tested in genetic models of cancer, including the MMTV-PyMT transgenic breast cancer model and the 5T2MM syngeneic tumor model of multiple myeloma. ALK1-Fc showed efficacy in inhibiting tumor progression in all models tested.

BMP9 expression was analyzed in various tumor types and it was found that BMP9 is expressed in breast cancer, cervical cancer, colorectal cancer, endometrial cancer, head and neck cancer, liver cancer, lung cancer, malignant carcinoma, malignant glioma, malignant lymphoma, malignant melanoma, ovarian cancer, and pancreatic cancer. These cancers are, accordingly, cancers that are responsive to treatment with an ALK1 antagonist as described herein. BMP9 expression is particularly high in colorectal cancer, head and neck cancer, liver cancer, and pancreatic cancer, making these cancers particularly suitable for treatment with an ALK1 antagonist according to methods provided herein.

Immunohistochemistry was used to determine the level of expression of BMP9 in 29 cases of head and neck cancer. Tumor biopsies were obtained and stained with an anti-BMP9 antibody in order to detect BMP9 expression. The observed levels of expression were classified into three categories 1+, 2+, and 3+, with 1+ indicating low expression, 2+ indicating medium expression, and 3+ indicating high expression. See FIG. 13 for exemplary immunohistochemistry images of different head and neck tumors of the larynx and tongue showing different levels of BMP9 expression. The results of the immunohistochemistry staining are summarized in Table 1:

TABLE 1
BMP9 expression levels based on IHC staining in 29 head and
neck tumor samples.

| CASE # | TISSUE | DIAGNOSIS | % | STRENGTH |
|---|---|---|---|---|
| 9836-F2 | EPIGLOTTIS | SCC | 100 | 3+ |
| 11913-A1 | EPIGLOTTIS | SCC | 90 | 3+ |
| 15923-A2 | HYPO- | SCC | 100 | 2+ |
| | PHARYNGEAL | | | |
| 6451-1 | LARYNGEAL | SCC | 40 | 2+ |
| ILS24458 | LARYNX | SCC | 50 | 2+ |
| 87-0082 2 | LARYNX | SCC | 80 | 3+ |
| 89-0244 5A | LARYNX | SCC | 30 | 1+ |
| 90-0118 | LARYNX | SCC | 50 | 2+ |
| 91-0204 | LARYNX | SCC | 60 | 2+ |
| 13192-D | LYMPHA- | SCC | 100 | 2-3+ |
| | DENOPATHY | | | |
| 91-0210 | NASOPHARYNX | SCC | 30 | 1+ |
| 17010 | PHARYNX | SCC | 50 | 2+ |
| 14860-B | PHARYNX | SCC | 50 | 2+ |
| 10083 | PHARYNX | SCC | 100 | 3+ |
| 3055-FSA1 | SOFT PALATE | SCC | 80 | 2+ |
| 87-00217 C3 | SOFT TISSUE | SCC | 90 | 3+ |
| 13917-A1FS | TONGUE | SCC | 50 | 1+ |
| 9227-C4 | TONGUE | SCC | 80 | 3+ |
| 3133-A4 | TONGUE | HG | 100 | 3+ |
| | | DYSPLASIA | | |
| 2188-A5 | TONGUE | SCC | 50 | 2+ |
| ILS27967 | TONGUE | SCC | 30 | 2+ |
| 89-0275 | TONGUE | SCC | 50 | 1+ |
| 1286 | TONGUE BX | SCC | 100 | 3+ |
| 10475-A2 | VOCAL CORD | SCC | 90 | 2-3+ |
| 4816-A | VOCAL CORD | SCC | 30 | 1+ |
| 8914-A | VOCAL CORD | SCC | 50 | 2+ |
| 89-0036 2 | VOCAL CORD | SCC | 60 | 2+ |
| 90-0301B | VOCAL CORD | SCC | 40 | 1+ |

| | | |
|---|---|---|
| 87-0217 D5 | Normal Larynx | 1+ of squamous epithelium, |
| | | 2+ of respiratory epithelium |
| 87-0255 B1 | Normal Esophagus | 3+ of basal layer, 0 of spinous layer |
| 89-0244 5I | Normal Larynx | 1+ of basal layer |
| 91-0168A | Normal Pharynx | no epithelium |
| 91-0175B | Normal Epiglottis | 1+ of basal layer, 2+ of |
| C5 | | respiratory epithelium |

Immunohistochemistry was used to determine the level of expression of BMP10 in the same 29 cases of head and neck cancer as described above in Table 4. Tumor biopsies were obtained and stained with an anti-BMP10 antibody in order to detect BMP10 expression. The observed levels of expression were classified into three categories: 1+, 2+, and 3+, with 1+ indicating low expression, 2+ indicating medium expression, and 3+ indicating high expression. The results of the immunohistochemistry staining are summarized in Table 2:

TABLE 2
BMP10 expression levels based on IHC staining in 29 head
and neck tumor samples

| CASE # | TISSUE | DIAGNOSIS | % | STRENGTH |
|---|---|---|---|---|
| 9836-F2 | EPIGLOTTIS | SCC | 50 | 3+ |
| 11913-A1 | EPIGLOTTIS | SCC | 0 | 0 |
| 15923-A2 | HYPOPHARYNGEAL | SCC | 30 | 2+ |
| 6451-1 | LARYNGEAL | SCC | 0 | 0 |
| ILS24458 | LARYNX | SCC | 0 | 0 |
| 87-0082 2 | LARYNX | SCC | 0 | 0 |
| 89-0244 5A | LARYNX | SCC | 0 | 0 |
| 90-0118 | LARYNX | SCC | 0 | 0 |
| 91-0204 | LARYNX | SCC | 70 | 3+ |
| 13192-D | LYMPHADENOPATHY | SCC | 0 | 0 |
| 91-0210 | NASOPHARYNX | SCC | 0 | 0 |
| 17010 | PHARYNX | SCC | 0 | 0 |
| 14860-B | PHARYNX | SCC | 70 | 3+ |
| 10083 | PHARYNX | SCC | 70 | 3+ |
| 3055-FSA1 | SOFT PALATE | SCC | 50 | 2+ |
| 87-00217 C3 | SOFT TISSUE | SCC | 30 | 1+ |
| 13917-A1FS | TONGUE | SCC | 0 | 0 |
| 9227-C4 | TONGUE | SCC | 90 | 3+ |
| 3133-A4 | TONGUE | HG DYS- | 70 | 3+ |
| | | PLASIA | | |
| 2188-A5 | TONGUE | SCC | 0 | 0 |
| ILS27967 | TONGUE | SCC | 0 | 0 |
| 89-0275 | TONGUE | SCC | 0 | 0 |
| 1286 | TONGUE BX | SCC | 30 | 2+ |
| 10475-A2 | VOCAL CORD | SCC | 40 | 2+ |
| 4816-A | VOCAL CORD | SCC | 0 | 0 |
| 8914-A | VOCAL CORD | SCC | 0 | 0 |
| 89-0036 2 | VOCAL CORD | SCC | 50 | 2+ |
| 90-0301B | VOCAL CORD | SCC | 0 | 0 |

The results, together show, that certain types of cancer express BMP9 and/or BMP10 and thus exhibit active ALK1 signaling. The cancers found positive for ALK1 agonists that are also characterized by vascularized tumors or that rely on or require angiogenesis for tumor survival, tumor cell proliferation, or tumor growth, are deemed responsive to treatment with an ALK1 antagonist described herein, e.g., an ALK1-ECD polypeptide or an ALK1-Fc fusion protein.

### Example 10

### Treatment of Cancer Expressing BMP9 and/or BMP10 with ALK1 Antagonists

Tumor samples were obtained from 29 subjects diagnosed with head and neck cancer. The tumor biopsies were subjected to immunohistochemistry analysis of BMP9 and BMP10 expression levels using anti-BMP9 and anti-BMP10 antibodies. Staining for each ALK1 agonist is quantified and classified into one of three categories: 1+, 2+, and 3+, with 1+ indicating low expression, 2+ indicating medium expression, and 3+ indicating high expression. The results of the quantification of BMP9 and BMP10 are shown in Tables 4 and 5.

Subjects are evaluated for their responsiveness to ALK1 antagonists based on the level of expression of BMP9 and BMP10 observed in the tumor samples.

In some embodiments, subjects showing an expression level of 3+ of either BMP9 or BMP10 in their tumor sample are identified as responsive to treatment with an ALK1-antagonist. For example, subjects 9836-F2 (BMP9: 3+, BMP10: 3+), 11913-A1 (BMP9: 3+), 87-0082 2 (BMP9: 3+), 91-0204 (BMP9: 2+, BMP10: 3+), 13192-D, 87-00217 C3, 9227-C4, 3133-A4, 1286, and 10475-A2 are identified, among others, to be responsive to ALK1 antagonist treatment. In some embodiments, an effective amount of an hALK1-Fc fusion protein is administered to these subjects to treat the head and neck cancer.

In some embodiments, subjects showing expression levels of 2+ of either BMP9 or BMP10 in their tumor sample are identified as responsive to treatment with an ALK1-antagonist. For example, subjects 9836-F2 (BMP9: 3+, BMP10: 3+), 11913-A1 (BMP9: 3+), 15923-A2 (BMP9: 2+, BMP10: 2+), 87-0082 2 (BMP9: 3+), 91-0204 (BMP9: 2+, BMP10: 3+), and 8914 A (BMP9: 2+) are identified, among others, to be responsive to ALK1 antagonist treatment. In some embodiments, an effective amount of an hALK1-Fc fusion protein is administered to these subjects to treat the head and neck cancer.

In some embodiments, subjects showing expression levels of 1+ of either BMP9 or BMP10 in their tumor sample are identified as responsive to treatment with an ALK1-antagonist. Accordingly, all subjects of Tables 4 and 5 are identified to be responsive to ALK1 antagonist treatment. In some embodiments, an effective amount of an hALK1-Fc fusion protein is administered to these subjects to treat the head and neck cancer.

In some embodiments, subjects showing expression levels of 1+ of BMP10 in their tumor sample are identified as responsive to treatment with an ALK1-antagonist. Accordingly, subjects 9836-F2 (BMP10: 3+), 15923-A2 (BMP10: 2+), 91-0204 (BMP10: 3+), 14860-B, 10083, 3055-FSA1, 87-00271C3, 9227-C4, 3133-A4, 1286, 10475-A2, and 89-0036 2 are identified to be responsive to ALK1 antagonist treatment. In some embodiments, an effective amount of an hALK1-Fc fusion protein is administered to these subjects to treat the head and neck cancer.

In some embodiments, those subjects expressing a level of BMP9 and/or of BMP10 in their respective tumor samples that is significantly higher than the level observed in a tissue sample obtained from healthy tissue, are selected for treatment with an ALK1 antagonist. In the case of the 29 subjects assessed, all subjects having a larynx tumor expressing a level of BMP9 of 2+ or higher in the basal layer or in the squamous epithelium, or of 3+ in respiratory epithelium; all subjects having an epiglottis tumor expressing a level of BMP9 of 2+ or higher in the basal layer, or of 3+ in the respiratory epithelium; and all subjects having an oesophagal tumor expressing a level of 1+ or higher of BMP9 in the spinous layer are selected (see the bottom of Table 4 for exemplary reference levels observed in healthy tissue).

In some embodiments, subjects are selected for treatment with an ALK1 antagonist based on the percentage of cells expressing BMP9 and/or BMP10 in their respective tumor sample. In some embodiments, subjects are indicated to be responsive to ALK1 antagonist treatment if their respective tumor samples show that more than 30%, more than 40%, more than 50%, more than 60%, more than 70%, more than 75%, more than 80%, more than 90%, more than 95%, or more than 98% of tumor cells express BMP9 and/or BMP10 as assessed by immunostaining.

### INCORPORATION BY REFERENCE

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes some embodiments in which exactly one member of the group is present in, employed in, or otherwise relevant to a given product or process. The invention also includes some embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus, for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A method for evaluating responsiveness of a subject to treatment with an activin receptor-like kinase 1 (ALK1) antagonist, the method comprising:
(a) determining a level of bone morphogenetic protein 9 (BMP9) and/or bone morphogenetic protein 10 (BMP10) in a sample obtained from the subject; and
(b) comparing the level of BMP9 and/or BMP 10 determined in (a) to a reference level, wherein
(i) if the level determined in (a) is higher than the reference level, the subject is identified as responsive to treatment with the ALK1 antagonist; or
(ii) if the level determined in (a) is the same or lower than the reference level, the subject is identified as not responsive to treatment with the ALK1 antagonist.

2. The method of claim 1, wherein the ALK1 antagonist comprises an agent selected from the group consisting of an ALK1-Fc fusion protein, an ALK1 extracellular domain (ALK-ECD), an antibody or antibody fragment specifically binding ALK1, an antibody or antibody fragment specifically binding an ALK1 ligand, an endoglin ECD antibody, an endoglin ECD, a BMP9 pro-peptide, and a BMP10 pro-peptide.

3. The method of claim 1, wherein the ALK1 antagonist comprises a polypeptide that is at least 95% identical to the polypeptide provided in SEQ ID NO: 3.

4. The method of claim 1, wherein the level of BMP9 and/or BMP10 is determined in a sample obtained from the subject.

5. The method of claim 4, wherein the sample is a tissue sample or body fluid sample.

6. The method of claim 5, wherein the tissue sample comprises a tumor tissue or a tumor cell.

7. (canceled)

8. The method of claim 1, wherein the level of BMP9 and/or BMP10 is determined by measuring the level of a BMP9 and/or BMP10 gene product.

9-10. (canceled)

11. The method of claim 1, wherein the subject is diagnosed with or is suspected to have a cancer.

12-14. (canceled)

15. A method of treating a subject with an ALK1 antagonist, the method comprising:
(a) selecting the subject for treatment with an ALK1 antagonist on the basis that the subject exhibits a level of BMP9 and/or BMP10 that is higher than a reference level; and
(b) administering the ALK1 antagonist to the subject.

16. The method of claim 15, wherein the subject is diagnosed with or is suspected to have a cancer.

17. The method of claim 16, wherein the cancer is breast cancer, multiple myeloma, cervical cancer, colorectal cancer, endometrial cancer, head and neck cancer, liver cancer, lung cancer, malignant carcinoid, malignant glioma, malignant lymphoma, malignant melanoma, ovarian cancer, or pancreatic cancer.

18. The method of claim 16, wherein the cancer is resistant to an angiogenesis inhibitor not comprising an ALK1 antagonist.

19. The method of claim 15, wherein the ALK1 antagonist comprises an agent selected from the group consisting of an ALK1-Fc fusion protein, an ALK1 extracellular domain (ALK-ECD), an antibody or antibody fragment specifically binding ALK1, an antibody or antibody fragment specifically binding an ALK1 ligand, an endoglin ECD antibody, an endoglin ECD, a BMP9 pro-peptide, and a BMP10 pro-peptide.

20. The method of claim 15, wherein the ALK1 antagonist comprises a polypeptide that is at least 95% identical to the polypeptide provided in SEQ ID NO: 3.

21. (canceled)

22. A diagnostic kit for evaluating responsiveness of a subject to treatment with an activin receptor-like kinase 1 (ALK1) antagonist, the kit comprising:
an agent for detecting a BMP9 and/or BMP10 gene product in a sample; and
instructions for detecting and/or quantifying a BMP9 and/or BMP 10 gene product.

23. The kit of claim 22, wherein the gene product is a protein.

24. The kit of claim 22, wherein the gene product is a transcript.

25. The kit of claim 22, wherein the agent is a binding agent that specifically binds the BMP9 and/or BMP 10 gene product.

26. The kit of claim 25 wherein the binding agent is an antibody or an antibody fragment that specifically bind the gene product.

27-29. (canceled)

30. The kit of claim 22, wherein the kit further comprises instructions for quantifying the level of BMP9 and/or BMP10.

31-52. (canceled)
